(19) United States
(12) Patent Application Publication
(10) Pub. No.: US 2005/0124660 A1
(43) Pub. Date: Jun. 9, 2005

(54) Novel medical uses of compounds showing CB1-antagonistic activity and combination treatment involving said compounds

(76) Inventors: Jochen Antel, Bad Munder (DE); Peter-Colin Gregory, Hannover (GB); Harald Waldeck, Isernhagen HB (DE); Gunter Krause, Burgdorf (DE); Josephus Hubertus Maria Lange, Almere (NL); Cornelis Gerrit Kruse, Amersfoort (NL)

Correspondence Address: Finnegan, Henderson, Farabow,;Garrett & Dunner, L.L.P., 1300 I Street, N.W., Washington, DC, 20005-3315, US

(21) Appl. No.: 10/969,840

(22) Filed: Oct. 22, 2004

Related U.S. Application Data
(60) Provisional application No. 60/513,995, filed on Oct. 27, 2003.

(51) Int. Cl. A61K 31/541; A61K 31/5377; A61K 31/454; A61K 31/4439; A61K 31/426; A61K 31/4196

(52) U.S. Cl. 514/326; 514/340; 514/370; 514/365; 514/383; 514/407; 514/210.2; 514/227.5; 514/235.2

(57) ABSTRACT

The present invention relates to a novel medical use of compounds with CB₁-receptor activity selected from the group of 4,5-dihydro-1H-pyrazole derivatives, 1H-Imidazole derivatives, thiazole derivatives and/or 1H-1,2,4-triazole-3-carboxamide derivatives, as each defined in the specification, or of a prodrug thereof, a tautomer thereof or a salt thereof, in the manufacture of medicaments for the treatment and/or prophylaxis of CB₁ receptor related diseases in juvenile patients and/or for the treatment and/or prophylaxis of drug induced obesity in juvenile, as well as in adolescent, patients. Furthermore, the invention pertains to the use of said compounds with CB₁-receptor activity in combination with lipase inhibitors. Said compounds are particularly suitable in combination with lipase inhibitors in the manufacture of medicaments for the treatment and/or prophylaxis of obesity in adolescent or in juvenile patients and/or for the treatment and/or prophylaxis of drug induced obesity in juvenile as well as in adolescent patients. Preferred lipase inhibitors are orlistat, panclicins, ATL-962 and/or lipstatin.

### CROSS REFERENCE TO RELATED APPLICATIONS

This application claims the benefit of U.S. Provisional Application No. 60/513,995, filed on Oct. 27, 2003, the contents of which are incorporated herein by reference.

The present invention relates to novel therapeutic and/or prophylactic uses of compounds showing CB₁-antagonistic activity and to pharmaceutical compositions containing one or more of these compounds as an active component for the novel uses. The compounds addressed in this invention show Cannabis-1 (CB₁) receptor antagonistic activity and may provide outstanding utility for the novel medical uses provided by the present invention. Furthermore, according to an embodiment of the present invention said compounds with CB₁-antagonistic activity may be used in combination with a further active principle, and to pharmaceutical compositions containing at least one of these CB₁-antagonistic compounds in combination with said further active principle for the treatment and/or prophylaxis of obesity. The combination provided by the present invention of said compounds having Cannabis-1 (CB₁) receptor antagonistic activity with said further active principle are of particular utility for treating of obesity.

Cannabinoids are present in the Indian hemp Cannabis Sativa L. and have been used as medicinal agents for centuries (Mechoulam, R.; Feigenbaum, J. J. Prog. Med. Chem. 1987, 24, 159). However, only within the past ten years the research in the cannabinoid area has revealed pivotal information on cannabinoid receptors and their (endogenous) agonists and antagonists. The discovery and the subsequent cloning of two different subtypes of Cannabinoid receptors (CB₁ and CB₂) stimulated the search for novel cannabinoid receptor antagonists (Munro, S.; Thomas, K. L.; Abu-Shaar, M. Nature 1993, 365, 61. Matsuda, L. A.; Bonner, T. I. Cannabinoid Receptors, Pertwee, R. G. Ed. 1995, 117, Academic Press, London). In addition, pharmaceutical companies became interested in the development of cannabinoid drugs for the treatment of diseases connected with disorders of the cannabinoid system. The wide distribution of CB₁ receptors in the brain, in combination with the strictly peripheral localization of the CB₂ receptor, makes the CB₁ receptor a very interesting molecular target for CNS-directed drug discovery in several areas of medical indications, e.g. psychiatric and neurological disorders are described in the state of the art as being of interest (Consroe, P. Neurobiology of Disease 1998, 5, 534. Pop, E. Curr. Opin. In CPNS Investigational Drugs 1999, 1, 587. Greenberg, D. A. Drug News Perspect. 1999, 12, 458).

From the international patent applications WO 03/026647, WO 03/027076, WO 03/078413, WO 03/078413, and the recently filed international patent application which is going to be published in March/April 2004 and which is based on the European priority application EP 02078966.5 with priority date Sep. 19, 2002, compounds are known, with utility for the treatment of diseases connected with disorders of the cannabinoid system. Thus, these compounds exhibit activity on the cannabis CB₁-receptor showing e.g. CB₁ antagonistic activity, and they have the formulas (I), (II), (III), (IV) or (V) as defined below in this specification. In particular said compounds with cannabis CB₁-receptor activity have been suggested for use in the treatment of psychiatric disorders such as psychosis, anxiety, depression, attention deficits, memory disorders, cognitive disorders, appetite disorders, obesity, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischemia, cerebral apoplexy, craniocerebral trauma, stroke, spinal cord injury, neuroinflammatory disorders, plaque sclerosis, viral encephalitis, demyelinisation related disorders, as well as for the treatment of pain disorders, including neuropathic pain disorders, and other diseases involving cannabinoid neurotransmission, including the treatment of septic shock, glaucoma, cancer, diabetes, emesis, nausea, asthma, respiratory diseases, gastrointestinal disorders, gastric ulcers, diarrhea and cardiovascular disorders.

The international patent application WO 03/026647 describes a group of novel compounds which are 4,5-dihydro-1H-pyrazole derivatives and have the formula (I) defined below. These 4,5-dihydro-1H-pyrazole derivatives are potent cannabinoid (CB₁) receptor antagonists with utility for the treatment of diseases connected with disorders of the cannabinoid system. The compounds have the general formula (I) wherein the symbols have the meanings given below in the specification, and show in particular potent CB₁ antagonistic activity.

The international patent application WO 03/027076 describes a group of novel compounds which are 1H-imidazole derivatives and have the formula (II) defined below. These 1H-imidazole derivatives are potent cannabinoid (CB₁) receptor agonists, partial agonists or antagonists, useful for the treatment of psychiatric and neurological disorders, as well as and other diseases involving cannabinoid neurotransmission. The compounds have the general formula (II) wherein the symbols have the meanings given below in the specification.

The international patent application WO 03/026648 describes a group of novel compounds which are also 4,5-dihydro-1H-pyrazole derivatives and have one of the formulas (III) defined below. These 4,5-dihydro-1H-pyrazole derivatives again are potent cannabinoid (CB1) receptor antagonists with utility for the treatment of diseases connected with disorders of the cannabinoid system. In particular, the compounds have the general formula (IIIa) or (IIIb) wherein the symbols have the meanings given in the specification below.

The international patent application WO 03/078413 describes a group of novel compounds which are thiazole derivatives and have of the formula (IV) defined below. These thiazole derivatives are potent antagonists, agonists or partial agonists of the cannabinoid CB1-receptor, with utility for the treatment of diseases connected with disorders of the cannabinoid system. The compounds have the general formula (IV) wherein the symbols have the meanings given in the specification below.

The recently filed international patent application, which is going to be published in March/April 2004 and which is based on the European priority application EP 02078966.5 with priority date 19.09.2002, describes a group of novel compounds which are 1,5-diaryl-1H-1,2,4-triazole-3-carboxamide derivatives and have of the formula (V) defined below. These 1,5-diaryl-1H-1,2,4-triazole-3-carboxamide derivatives are potent antagonists, agonists, inverse agonists or partial agonists of the cannabinoid CB₁ receptor, with utility for the treatment of diseases connected with disorders of the cannabinoid system. The compounds have the general formula (V) wherein the symbols have the meanings given in the specification below.

It is an objective of the invention to provide improved methods of treatment and/or prophylaxis which are particularly suitable in patient groups with enhanced need of safety and tolerability, e.g. in the treatment of obesity patients, in particular such as juvenile obesity patients and/or patients subject to long term treatment, e.g. in drug induced obesity in juvenile or adolescent patients. A further objective is to provide particular beneficial combination treatments and medicaments therefore for the treatment and/or prophylaxis of obesity in patients of any age, e.g. in adolescent as well as in juvenile patients, wherein the compounds with CB₁ antagonistic activity used according to the present invention are combined with a further active principle for the treatment of obesity.

It has now surprisingly been found that due to the outstanding unique pharmacological profile of selective CB₁-antagonistic compounds which includes particularly high safety and tolerability the compounds particularly suitable for treatments and/or prophylaxis of diseases connected with disorders of the cannabinoid system, in particular in patient groups with enhanced need of safety and tolerability, in particular such as juvenile patients and/or patients subject to long term treatment, e.g. in drug induced obesity.

Therefore, the invention pertains to a combination of a compound with CB₁-receptor activity having one of the formulas (I), (II), (III), (IV), or (V) as subsequently defined, or a prodrug, tautomer or salt thereof, preferably of a CB₁ receptor antagonistic compound or a prodrug, tautomer or salt thereof, with at least one lipase inhibiting compound. In a variant of the invention, the compound with CB₁-receptor activity having one of the formulas (I), (II), (III), (IV), or (V) as subsequently defined, or a prodrug, tautomer or salt thereof, preferably the CB₁ receptor antagonistic compound or a prodrug, tautomer or salt thereof, is in combination with at least one lipase inhibiting compound selected from the group of lipase inhibiting polymers, orlistat, paclicins, ATL-962 and lipstatin.

In particular, it has surprisingly been found that the CB₁-antagonists of formulas (I), (II), (III), (IV) and/or (V), prodrugs thereof, tautomers thereof and salts thereof, show a unique pharmacological profile and therefore are particularly suited for the use in the manufacture of a medicaments for the treatment and/or prophylaxis of obesity patients, in particular of obesity in juvenile patients and/or drug induced obesity in juvenile, as well as adolescent, patients. In this regard the CB₁-antagonistic compounds of formulas (I), (II), (III), (IV) and/or (V), prodrugs thereof, tautomers thereof and salts thereof, are highly valuable in providing medicaments for pediatric use on the one hand, and for the general use in drug induced obesity.

Prodrugs are bioreversible derivatives of drug molecules used to overcome some barriers to the utility of the parent drug molecule. These barriers include, but are not limited to, solubility, permeability, stability, presystemic metabolism and targeting limitations (J. Stella, “Prodrugs as therapeutics”, Expert Opin. Ther. Patents, 14(3), 277-280, 2004). Pro-drugs, i.e., compounds which when administered to humans by any known route, are metabolized to compounds having a formula set forth herein, belong to the invention. For example, this includes compounds with primary or secondary amino or hydroxy groups. Such compounds can be reacted with organic acids to yield compounds having a formula set forth herein wherein an additional group is present which is easily removed after administration, for instance, but not limited to amidine, enamine, a Mannich base, a hydroxylmethylene derivative, an O-(acyloxymethylene carbamate) derivative, carbamate, ester, amide or enaminone. A pro-drug is an inactive compound, which when administered is converted into an active form. See Medicinal Chemistry: Principles and Practice, 1994, ISBN 0-85186-494-5, Ed.: F. D. King, p. 215.

As stated above, the compounds of the present invention can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Due to their activity on the cannabis CB₁-receptor the compounds used according to the invention are suitable also for use in pediatric treatment and/or prophylaxis of other disorders than juvenile obesity and drug induced obesity in juvenile patients. The other disorders include those known from the literature for the concerned compounds with activity on the cannabis CB₁-receptor, and e.g. the pediatric treatment and/or prophylaxis may also pertain to psychiatric disorders such as psychosis, anxiety, depression, attention deficits, memory disorders, cognitive disorders, obesity, disorders, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischemia, cerebral apoplexy, craniocerebral trauma, stroke, spinal cord injury, neuroinflammatory disorders, plaque sclerosis, viral encephalitis, demyelinisation related disorders, as well as for the pediatric treatment of pain disorders, including neuropathic pain disorders, and other diseases involving cannabinoid neurotransmission, including the pediatric treatment of septic shock, glaucoma, cancer, diabetes, emesis, nausea, asthma, respiratory diseases, gastrointestinal disorders, gastric ulcers, diarrhoea and cardiovascular disorders, in young patients.

The whole content of the literature mentioned in the description of the present invention is incorporated by reference into the present application.

The CB₁ antagonistic compounds used in the present the invention can be obtained according to known methods. Suitable ways of synthesis for the compounds used according to the present invention are described in the state of the art, e.g. in the documents cited in the present application and incorporated by reference.

Furthermore, it has surprisingly been found that CB₁ antagonistic compounds (CB₁ antagonists), of the formulas (I), (II), (III), (IV) and/or (V), as well as prodrugs, tautomers and salts thereof, due to their unique pharmacological profile are particularly suited in combination with at least one lipase inhibiting compound (lipase inhibitor) for the use in the manufacture of a medicaments for the treatment and/or prophylaxis of obesity, including in particular the treatment and/or prophylaxis of obesity in juvenile patients and/or drug induced obesity in juvenile as well as adolescent patients. In this regard combinations of at least one CB₁ antagonistic compound as defined herein with at least one lipase inhibiting compound are highly valuable in providing medicaments for the treatment and/or prophylaxis of obesity in general, e.g. in adolescent patients of any age, and particularly also in pediatric or juvenile obesity, and also in drug induced obesity in adolescent and juvenile patients.

In particular the present invention is based on the surprising finding that the CB₁ antagonistic compounds of the formulas (I) being 4,5-dihydro-1H-pyrazole derivatives, (II) being 1H-Imidazole derivatives, (III) being also 4,5-dihydro-1H-pyrazole derivatives, (IV) being thiazole derivatives or (V) being 1H-1,2,4-triazole-3-carboxamide derivatives, which are antagonists of the cannabis CB₁-receptor, prodrugs thereof, tautomers thereof and salts thereof, due to their unique pharmacological profile are particularly suited in combination with at least one lipase inhibiting compound for the use in the manufacture of a medicament for the treatment and/or prophylaxis of obesity in general, e.g. of obesity in adolescent patients of any age, and particularly also for the treatment and/or prophylaxis of obesity in juvenile patients and/or drug induced obesity in juvenile as well as adolescent patients. In this regard combinations each of the compounds with the formulas (I), (II), (III), IV or (V), together with lipase inhibiting compounds are highly valuable in providing medicaments for the treatment and/or prophylaxis of obesity in general, e.g. of obesity in adolescent patients of any age, and particularly also in pediatric or juvenile obesity, and in drug induced obesity.

The compounds of the formulas (I), (II), (III), (IV) or (V) used in the present the invention can be obtained according to known methods. A suitable synthesis for the compounds of the formulas (I), (II), (III), (IV) used according to the present invention is described in the international patent applications WO 03/026647, WO 03/027076, WO 03/078413 or WO 03/078413, which are incorporated by reference into this specification. Compounds of formula (V) can be prepared according to a recently filed international patent application, which is going to be published in March/April 2004 and is based on the European priority application EP 02078966.5 with priority date Sep. 19, 2002, which is also incorporated by reference into the present specification. Preparation of compounds of formula (V) is also described at the end of this specification.

In the following the embodiments of the invention are described in more detail with reference to the compounds with the formulas (I), (II), III), IV or (V), exemplary in particular in the context of obesity.

Compounds of Formula (I)

In a first embodiment the present invention is based on the surprising finding that the 4,5-dihydro-1H-pyrazole derivatives of the formula (I) which are potent and selective antagonists of the cannabis CB₁-receptor, prodrugs thereof, tautomers thereof and salts thereof: 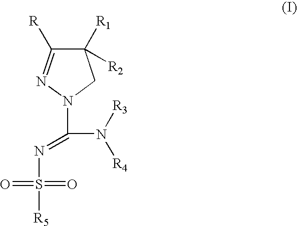
wherein
R and R₁ independently represent phenyl, thienyl or pyridyl which groups may be substituted with 1, 2, 3 or 4 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
R₃ represents a hydrogen atom or a branched or unbranched C₁₋₈ alkyl group or a C₃₋₇ cycloalkyl group which alkyl group or cycloalkyl group may be substituted with a hydroxy group,
R₄ represents a C₂₋₁₀ branched or unbranched heteroalkyl group, C₃₋₈ non-aromatic heterocycloalkyl group or C₄₋₁₀ non-aromatic heterocycloalkyl-alkyl group which groups contain one or more heteroatoms from the group (O, N, S) or a —SO₂— group, which C₂₋₁₀ branched or unbranched heteroalkyl group, C₃₋₈ non-aromatic heterocycloalkyl group or C₄₋₁₀ non-aromatic heterocycloalkyl-alkyl group may be substituted with a keto group, trifluoromethyl group, C₁₋₃ alkyl group, hydroxy, amino, monoalkylamino, or dialkylamino group or a fluoro atom, or R₄ represents an amino, hydroxy, phenoxy or benzyloxy group, or R₄ represents a C₁₋₈ alkoxy, C₃₋₈ alkenyl, C₅₋₈ cycloalkenyl or C₆₋₉ cycloalkenylalkyl group which groups may contain a sulphur, nitrogen or oxygen atom, a keto group or —SO₂— group, which alkoxy, alkenyl and cycloalkenyl groups may be substituted with a hydroxy group, a trifluoromethyl group, an amino group, a monoalkylamino group or dialkylamino group or a fluoro atom, or R₄ represents a C₂₋₅ alkyl group which alkyl group contains a fluoro atom, or R₄ represents an imidazolylalkyl group, benzyl, pyridylmethyl, phenethyl or thienyl group, or R₄ represents a substituted phenyl, benzyl, pyridyl, thienyl, pyridylmethyl or phenethyl group wherein the aromatic rings are substituted with 1, 2 or 3 of the substituents Y, wherein Y has the meaning as indicated above, or when R₃ is H or methyl, R₄ may represent a group NR₆R₇ wherein
R₆ and R₇ are the same or different and represent C₂₋₄ alkyl, C₂₋₄ trifluoroalkyl or R₆ represents a methyl group with the proviso that R₇ represents a C₂₋₄ alkyl group, or R₆ and R₇—together with the nitrogen atom to which they are bonded—form a saturated or unsaturated heterocyclic moiety having 4 to 8 ring atoms which heterocyclic moiety may contain an oxygen or sulphur atom or a keto group or —SO₂— group or an additional nitrogen atom, which saturated or unsaturated heterocyclic moiety may be substituted with a C₁₋₄ alkyl group, or
R₃ and R₄ together with the nitrogen atom to which they are bonded form a saturated or unsaturated, monocyclic or bicyclic heterocyclic moiety having 4 to 10 ring atoms, which heterocyclic moiety may contain one or more atoms from the group (O, N, S) or a keto group or —SO₂— group, which moiety may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group,
R₅ represents benzyl, phenyl, thienyl or pyridyl which may be substituted with 1, 2, 3 or 4 substituents Y, wherein Y has the meaning as indicated above, which can be the same or different, or R₅ represents C₁₋₈ branched or unbranched alkyl, C₃₋₈ alkenyl, C₃₋₁₀ cycloalkyl, C₅₋₁₀ bicycloalkyl, C₆₋₁₀ tricycloalkyl or C₅₋₈ cycloalkenyl or R₅ represents naphtyl,
   due to their unique pharmacological profile are particularly suited in combination with at least one lipase inhibiting compound for the use in the manufacture of a medicament for the treatment and/or prophylaxis of obesity in general, e.g. of obesity in adolescent patients of any age, and particularly also for the treatment and/or prophylaxis of obesity in juvenile patients and/or drug induced obesity in juvenile as well as adolescent patients. In this regard combinations of the compounds of formula (I) together with lipase inhibiting compounds are highly valuable in providing medicaments for the treatment and/or prophylaxis of obesity in general, e.g. of obesity in adolescent patients of any age, and particularly also in pediatric or juvenile obesity, and in drug induced obesity.

At least one centre of chirality is present (at the C₄ position of the 4,5-dihydro-1H-pyrazole moiety) in the compounds of the formula (I). The invention relates both to racemates, mixtures of diastereomers and the individual stereoisomers of the compounds having formula (I). Particular compounds of interest of formula (I) have the absolute stereoconfiguration at the C₄ position of the 4,5-dihydro-1H-pyrazole moiety as represented by formula (1^{a}).

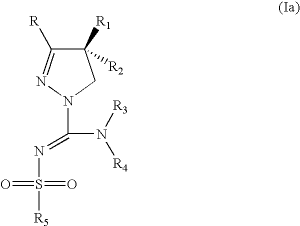

The invention also relates both to the E isomer, Z isomer and E/Z mixtures of compounds having formula (I).

Compounds of Formula (II)

In a second embodiment the present invention is based on the surprising finding that the 1H-imidazole derivatives with CB₁ antagonistic activity of the formula (II), prodrugs thereof and salts thereof, which are potent antagonists on cannabinoid-CB₁ receptors: 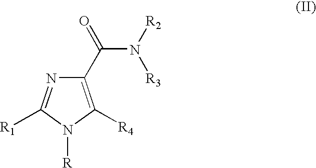
wherein
R represents phenyl, thienyl, 2-pyridinyl, 3-pyridinyl, 4-pyridinyl, pyrimidinyl, pyrazinyl, pyridazinyl or triazinyl, which groups may be substituted with 1, 2, 3 or 4 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkoxycarbonyl, carboxyl, cyano, carbamoyl and acetyl, or R represents naphtyl, with the proviso that when R is 4-pyridinyl, R₄ represents a halogen atom or a cyano, carbamoyl, formyl, acetyl, trifluoroacetyl, fluoroacetyl, propionyl, sulfamoyl, methanesulfonyl, methylsulfanyl or branched or unbranched C₁₋₄ alkyl group, which C₁₋₄ alkyl group may be substituted with 1-3 fluoro atoms or with a bromo, chloro, iodo, cyano or hydroxy group,
R₁ represents phenyl or pyridinyl, which groups may be substituted with 1-4 substituents Y, which can be the same or different, wherein Y has the above mentioned meaning, or R₁ represents pyrimidinyl, pyrazinyl, pyridazinyl or triazinyl, which groups may be substituted with 1-2 substituents Y, which can be the same or different or R₁ represents a five-membered aromatic heterocyclic ring having one or two heteroatoms from the group (N, O, S), which heteroatoms can be the same or different, which five-membered aromatic heterocyclic ring may be substituted with 1-2 substituents Y, which can be the same or different or R₁ represents naphtyl,
R₂ represents H, branched or unbranched C₁₋₈ alkyl, C₃₋₈ cycloalkyl, C₃₋₈ alkenyl, C₅₋₈ cycloalkenyl which groups may contain a sulfur, oxygen or nitrogen atom,
R₃ represents branched or unbranched C₂₋₈ alkyl, C₁₋₈ alkoxy, C₅₋₈ cycloalkyloxy, C₃₋₈ cycloalkyl, C₅₋₁₀ bicycloalkyl, C₆₋₁₀ tricycloalkyl, C₃₋₈ alkenyl, C₅₋₈ cycloalkenyl, which groups may optionally contain one or more heteroatoms from the group (O, N, S) and which groups may be substituted with a hydroxy group or 1-2 C₁₋₃ alkyl groups or 1-3 fluoro atoms, or R₃ represents a benzyl or phenethyl group which aromatic rings may be substituted with 1-5 substituents Z, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkylsulfonyl, dimethyl-sulfamido, C₁₋₃-alkoxycarbonyl, carboxyl, trifluoromethylsulfonyl, cyano, carbamoyl, sulfamoyl and acetyl, or R₃ represents a phenyl or pyridinyl group, which groups are substituted with 1-4 substituents Z, wherein Z has the meaning as indicated above, or R₃ represents a pyridinyl group, or R₃ represents a phenyl group, with the proviso that R₄ represents a halogen atom or a cyano, carbamoyl, formyl, acetyl, trifluoroacetyl, fluoroacetyl, propionyl, sulfamoyl, methanesulfonyl, methylsulfanyl or C₁₋₄ alkyl group, which C₁₋₄ alkyl group may be substituted with 1-3 fluoro atoms or with a bromo, chloro, iodo, cyano or hydroxy group, or R₃ represents a group NR₅R₆ with the proviso that R₂ represents a hydrogen atom or a methyl group, wherein
R₅ and R₆ are the same or different and represent branched or unbranched C₁₋₄ alkyl, or R₅ and R₆—together with the nitrogen atom to which they are bonded—form a saturated or unsaturated, monocyclic or bicyclic heterocyclic group having 4 to 10 ring atoms which heterocyclic group contains one or two heteroatoms from the group (N, O, S), which heteroatoms can be the same or different, which heterocyclic group may be substituted with a C₁₋₃ alkyl group or a hydroxy group, or R₂ and R₃—together with the nitrogen atom to which they are bonded—form a saturated or unsaturated heterocyclic group having 4 to 10 ring atoms which heterocyclic group contains one or two heteroatoms from the group (N, O, S), which heteroatoms can be the same or different, which heterocyclic group may be substituted with a C₁₋₃ alkyl group or a hydroxy group,
R₄ represents a hydrogen or halogen atom or a cyano, carbamoyl, formyl, acetyl, trifluoroacetyl, fluoroacetyl, propionyl, sulfamoyl, methanesulfonyl, methylsulfanyl or branched or unbranched C₁₋₄ alkyl group, which C₁₋₄ alkyl group may be substituted with 1-3 fluoro atoms or with a bromo, chloro, iodo, cyano or a hydroxy group,
   due to their unique pharmacological profile are particularly suited in combination with at least one lipase inhibiting compound for the use in the manufacture of a medicament for the treatment and/or prophylaxis of obesity in general, e.g. of obesity in adolescent patients of any age, and particularly also for the treatment and/or prophylaxis of obesity in juvenile patients and/or drug induced obesity in juvenile as well as adolescent patients. In this regard combinations of the compounds of formula (II) together with lipase inhibiting compounds are highly valuable in providing medicaments for the treatment and/or prophylaxis of obesity in general, e.g. of obesity in adolescent patients of any age, and particularly also in pediatric or juvenile obesity, and in drug induced obesity.
   Compounds of Formula (III)

In a third embodiment the present invention is based on the surprising finding that potent and selective antagonism of cannabinoid-CB₁ receptors is present in the 4,5-dihydro-1H-pyrazole derivatives of the formula (IIIa) or (IIIb), prodrugs thereof, tautomers thereof and salts thereof: 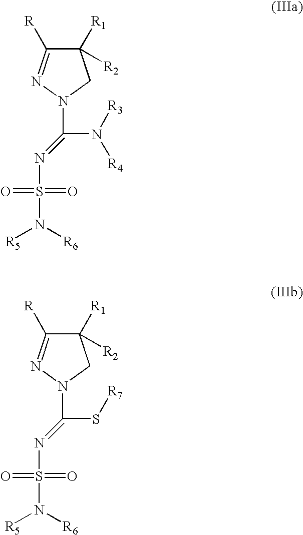
wherein
R and R₁ independently represent phenyl, thienyl or pyridyl which groups may be substituted with 1, 2 or 3 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
R₃ represents a hydrogen atom or a branched or unbranched C₁₋₈ alkyl group or a C₃₋₇ cycloalkyl group which alkyl group or cycloalkyl group may be substituted with a hydroxy group,
R₄ represents a hydrogen atom or a branched or unbranched C₁₋₈ alkyl, C₃₋₈ cycloalkyl, C₂₋₁₀ heteroalkyl, C₃₋₈ nonaromatic heterocycloalkyl or C₄₋₁₀ nonaromatic heterocycloalkyl-alkyl moiety which moieties may contain one or more heteroatoms from the group (O, N, S), which moieties may be substituted with a keto group, trifluoromethyl group, C₁₋₃ alkyl group, hydroxy, amino, monoalkylamino, or dialkylamino group or a fluoro atom, or R₄ represents an amino, hydroxy, phenoxy or benzyloxy group or R₄ represents a branched or unbranched C₁₋₈ alkoxy, C₃₋₈ alkenyl, C₅₋₈ cycloalkenyl or C₆₋₉ cycloalkenylalkyl group which groups may contain a sulphur, nitrogen or oxygen atom, a keto group or —SO₂— group which C₁₋₈ alkoxy, C₃₋₈ alkenyl, C₅₋₈ cycloalkenyl or C₆₋₉ cycloalkenylalkyl groups may be substituted with a hydroxy group, a trifluoromethyl group, an amino group, a monoalkylamino group or dialkylamino group or a fluoro atom, or R₄ represents a phenyl, benzyl, pyridyl, thienyl, pyridylmethyl or phenethyl group wherein the aromatic rings may be substituted with 1, 2 or 3 of the substituents Y, wherein Y has the meaning as indicated above, or R₄ represents a group NR₈R₉ with the proviso that R₃ represents a hydrogen atom or a methyl group and wherein R₈ and R₉ are the same or different and represent C₁₋₄ alkyl or C₂₋₄ trifluoroalkyl or R₈ and R₉—together with the nitrogen atom to which they are bonded—form a saturated or un-saturated heterocyclic moiety having 4 to 8 ring atoms which heterocyclic moiety may contain an oxygen or sulphur atom or a keto group or —SO₂— group or an additional nitrogen atom, which saturated or unsaturated heterocyclic moiety may be substituted with a C₁₋₄ alkyl group or R₃ and R₄—together with the nitrogen atom to which they are bonded—form a saturated or unsaturated, monocyclic or bicyclic heterocyclic moiety having 4 to 10 ring atoms, which heterocyclic moiety may contain one or more atoms from the group (O, N, S) or a keto group or —SO₂— group, which moiety may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group,
R₅ and R₆ independently of each other represent a hydrogen atom or a branched or unbranched C₁₋₈ alkyl or alkenyl group which groups may contain one or more heteroatoms from the group (O, N, S), a keto group or a —SO₂— group and which groups may be substituted with a hydroxy or amino group, or R₅ and R₆ independently of each other represent a C₃₋₈ cycloalkyl group or C₃₋₈ cycloalkenyl group which may contain one or more ring heteroatoms from the group (O, N, S) or the —SO₂— group and which groups may be substituted with a hydroxy group, alkyl (C₁₋₃), the —SO₂— group, the keto group, amino group, monoalkylamino group (C₁₋₃) or dialkylamino group (C₁₋₃), or R₅ represents a naphtyl group or a phenyl group which phenyl group may be substituted with 1, 2 or 3 substituents Y wherein Y has the meaning as described hereinabove, with the proviso that R₆ represents a hydrogen atom, or a branched or unbranched alkyl group (C₁₋₅) which alkyl group may contain one or more heteroatoms from the group (O, N, S) or the —SO₂— group and which alkyl group may be substituted with a hydroxy, keto or amino group, or R₅ and R₆— together with the nitrogen atom to which they are bonded—form a monocyclic, bicyclic or tricyclic alkyl or alkenyl group which may contain ring heteroatoms from the group (O, N, S), the keto or the SO₂ group and which monocyclic, bicyclic or tricyclic alkyl or alkenyl group may be substituted with a hydroxy group, alkyl (C₁₋₃) group, SO₂ group, keto group, amino group, monoalkylamino group (C₁₋₃), dialkylamino group (C₁₋₃), pyrrolidinyl group or piperidinyl group, which monocyclic, bicyclic or tricyclic alkyl or alkenyl group may contain an annelated phenyl group which annelated phenyl group may be substituted with 1 or 2 substituents Y, wherein Y has the meaning as described herein above,
R₇ represents branched or unbranched C₁₋₃ alkyl,
   due to their unique pharmacological profile are particularly suited in combination with at least one lipase inhibiting compound for the use in the manufacture of a medicament for the treatment and/or prophylaxis of obesity in general, e.g. of obesity in adolescent patients of any age, and particularly also for the treatment and/or prophylaxis of obesity in juvenile patients and/or drug induced obesity in juvenile as well as adolescent patients. In this regard combinations of the compounds of formula (IIIa) and/or (IIIb) together with lipase inhibiting compounds are highly valuable in providing medicaments for the treatment and/or prophylaxis of obesity in general, e.g. of obesity in adolescent patients of any age, and particularly also in pediatric or juvenile obesity, and in drug induced obesity.

At least one centre of chirality is present (at the C₄ position of the 4,5-dihydro-1H-pyrazole moiety) in the compounds of the formula (IIIa) and (IIIb). The invention relates both to racemates, mixtures of diastereomers and the individual stereoisomers of the compounds having formula (IIIa) or (IIIb). Particular compounds of interest of formula (IIIa) or (IIIb) have the absolute stereoconfiguration at the C₄ position of the 4,5-dihydro-1H-pyrazole moiety as represented by the formulas (IIIa*) and (IIIb*):

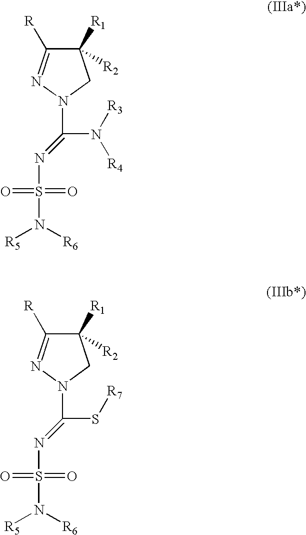

The invention also relates both to the E isomer, Z isomer and E/Z mixtures of compounds having formula (IIIa) or (IIIb).

Compounds of Formula (IV)

In a fourth embodiment the present invention is based on the surprising finding that the 4,5-diarylthiazole derivatives with CB₁ antagonistic activity of the formula (IV), pro-drugs thereof and salts thereof 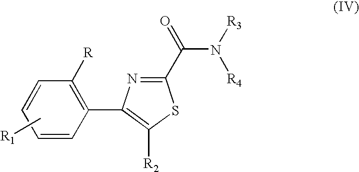
wherein
R represents a hydrogen atom or a substituent X from the group branched or unbranched C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, branched or unbranched (C₁₋₃)-alkoxycarbonyl, trifluoromethylsulfonyl, sulfamoyl, branched or unbranched alkyl(C₁₋₃)sulfonyl, carboxyl, cyano, carbamoyl, branched or unbranched dialkyl(C₁₋₃) aminosulfonyl, branched or unbranched monoalkyl(C₁₋₃)-aminosulfonyl and acetyl,
R₁ is a hydrogen atom or represents 1-4 substituents X, wherein X has the abovementioned meaning,
R₂ represents a phenyl, thienyl, pyridyl or pyrimidinyl group, which groups may be substituted with 1-4 substituents X, wherein X has the abovementioned meaning or R₂ represents naphtyl,
R₃ represents a hydrogen atom or a branched or unbranched C₁₋₁₀ alkyl or cycloalkyl-alkyl group or a phenyl, benzyl or phenethyl group which aromatic rings may be substituted with 1-5 substituents Z, which can be the same or different, from the group branched or unbranched C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, branched or unbranched (C₁₋₃)-alkylsulfonyl, dimethylsulfamido, branched or unbranched C₁₋₃-alkoxycarbonyl, carboxyl, trifluoromethylsulfonyl, cyano, carbamoyl, sulfamoyl and acetyl, or R₃ represents a pyridyl or thienyl group,
R₄ represents branched or unbranched C₁₋₁₀ alkyl or cycloalkyl-alkyl group, branched or unbranched C₁₋₁₀ alkoxy, C₃₋₈ cycloalkyl, C₅₋₁₀ bicycloalkyl, C₆₋₁₀ tricycloalkyl, branched or unbranched C₃₋₁₀ alkenyl, C₅₋₈ cycloalkenyl, which groups may contain one or more heteroatoms from the group (O, N, S) and which groups may be substituted with a hydroxy group, 1-3 methyl groups, an ethyl group or 1-3 fluoro atoms, or R₄ represents a phenyl, benzyl or phenethyl group which aromatic rings may be substituted with 1-5 substituents Z, wherein Z has the abovementioned meaning, or R₄ represents a pyridyl or thienyl group, or R₄ represents a group NR₅R₆ wherein R₅ and R₆ together with the nitrogen atom to which they are attached form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or unbranched C₁₋₃ alkyl, hydroxy or trifluoromethyl group or a fluoro atom, or
R₃ and R₄—together with the nitrogen atom to which they are attached—form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or unbranched C₁₋₃ alkyl, hydroxy or trifluoromethyl group or a fluoro atom,
   due to their unique pharmacological profile are particularly suited in combination with at least one lipase inhibiting compound for the use in the manufacture of a medicament for the treatment and/or prophylaxis of obesity in general, e.g. of obesity in adolescent patients of any age, and particularly also for the treatment and/or prophylaxis of obesity in juvenile patients and/or drug induced obesity in juvenile as well as adolescent patients. In this regard combinations of the compounds of formula (IV) together with lipase inhibiting compounds are highly valuable in providing medicaments for the treatment and/or prophylaxis of obesity in general, e.g. of obesity in adolescent patients of any age, and particularly also in pediatric or juvenile obesity, and in drug induced obesity.
   Compounds of Formula (V)

In a fifth embodiment the present invention is based on the surprising finding that 1,5-diaryl-1H-1,2,4-triazole-3-carboxamide derivatives with CB₁ antagonistic activity of the formula (V), as well as prodrugs, salts, and stereo-isomers thereof, are potent antagonists, agonists, inverse agonists or partial agonists of the cannabinoid CB₁ receptor: 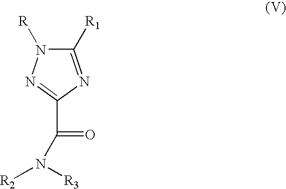
wherein
   R and R₁ independently represent a phenyl, naphtyl, thienyl, pyridyl, pyrimidyl, pyrazinyl, pyridazinyl or triazinyl group, which groups may be substituted with 1-4 substituents X, which can be the same or different, from the group branched or unbranched (C₁₋₃)-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkoxycarbonyl, trifluoromethylsulfonyl, sulfamoyl, (C₁₋₃)-alkylsulfonyl, carboxyl, cyano, carbamoyl, (C₁₋₃)-dialkylaminosulfonyl, (C₁₋₃)-monoalkylamino-sulfonyl and acetyl,
   R₂ represents a hydrogen atom or a branched or unbranched C₁₋₈ alkyl or C₁₋₈ cycloalkyl-alkyl group or a phenyl, benzyl or phenethyl group which aromatic rings may be substituted with 1-4 substituents X, wherein X has the meaning as indicated above, or R₂ represents a pyridyl or thienyl group,
   R₃ represents branched or unbranched C₁₋₈ alkyl, C₁₋₈ alkoxy, C₃₋₈ cycloalkyl, C₅₋₁₀ bicycloalkyl, C₆₋₁₀ tricycloalkyl, C₃₋₈ alkenyl, C₅₋₈ cycloalkenyl, which groups may optionally contain one or more heteroatoms from the group (O, N, S), which groups may be substituted with a hydroxy group, an ethynyl group or 1-3 fluoro atoms, or R₃ represents a phenyl, benzyl or phenethyl group which aromatic rings may be substituted with 1-4 substituents X, wherein X has the meaning as indicated above, or R₃ represents a pyridyl, pyrimidyl, pyrazinyl, pyridazinyl, triazinyl or thienyl group which heteroaromatic rings may be substituted with 1-2 substituents X, wherein X has the meaning as indicated above, or R₃ represents a group NR₄R₅ wherein R₄ and R₅, together with the nitrogen atom to which they are bonded, form a saturated or unsaturated, monocyclic or bicyclic, heterocyclic moiety having 4 to 10 ring atoms, which heterocyclic group contains one or two heteroatoms from the group N, O or S, which heteroatoms can be the same or different, which heterocyclic moiety may be substituted with a branched or unbranched C₁₋₃ alkyl, hydroxy or trifluoromethyl group or a fluoro atom, or
   R₂ and R₃, together with the nitrogen atom to which they are bonded, form a saturated or unsaturated, monocyclic or bicyclic, heterocyclic moiety having 4 to 10 ring atoms, which heterocyclic group contains one or two heteroatoms from the group N, O or S, which heteroatoms can be the same or different, which heterocyclic moiety may be substituted with a branched or unbranched C₁₋₃ alkyl, hydroxy, piperidinyl or trifluoromethyl group or a fluoro atom,
      due to their unique pharmacological profile are particularly suited in combination with at least one lipase inhibiting compound for the use in the manufacture of a medicament for the treatment and/or prophylaxis of obesity in general, e.g. of obesity in adolescent patients of any age, and particularly also for the treatment and/or prophylaxis of obesity in juvenile patients and/or drug induced obesity in juvenile as well as adolescent patients. In this regard combinations of the compounds of formula (V) together with lipase inhibiting compounds are highly valuable in providing medicaments for the treatment and/or prophylaxis of obesity in general, e.g. of obesity in adolescent patients of any age, and particularly also in pediatric or juvenile obesity, and in drug induced obesity.

In the following the embodiments of the invention are described in more detail with reference to the medical and pharmaceutical utility of the compounds with the formulas (I), (I), III), IV or (V).

Medical and Pharmaceutical Utility

Due to their beneficial activity on the cannabis CB₁-receptor the compounds of formulas (I), (II), (III), (IV) and/or (V) used according to the invention are suitable also for general use in pediatric treatment and/or prophylaxis of other disorders than juvenile obesity and drug induced obesity in juvenile patients. The other disorders include those known from the literature for the concerned compounds with activity on the cannabis CB₁-receptor, and e.g. the pediatric treatment and/or prophylaxis may also pertain to psychiatric disorders such as psychosis, anxiety, depression, attention deficits, memory disorders, cognitive disorders, appetite disorders, obesity, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischemia, cerebral apoplexy, craniocerebral trauma, stroke, spinal cord injury, neuroinflammatory disorders, plaque sclerosis, viral encephalitis, demyelinisation related disorders, as well as for the pediatric treatment of pain disorders, including neuropathic pain disorders, and other diseases involving cannabinoid neurotransmission, including the pediatric treatment of septic shock, glaucoma, cancer, diabetes, emesis, nausea, asthma, respiratory diseases, gastrointestinal disorders, gastric ulcers, diarrhoea and cardiovascular disorders, in young patients.

The affinity of the compounds of the formulas (I), (II), III), (IV) or (V) for cannabinoid CB₁ receptors can be determined as described in the WO 03/026647, WO 03/027076, WO 03/026648 or WO 03/078413, e.g. it can be determined using membrane preparations of Chinese hamster ovary (CHO) cells in which the human cannabis CB₁ receptor is stably transfected in conjunction with [3H]CP-55,940 as radioligand. After incubation of a freshly prepared cell membrane preparation with the [3H]-ligand, with or without addition of compounds of the invention, separation of bound and free ligand was performed by filtration over glass fibre filters. Radioactivity on the filter was measured by liquid scintillation counting.

The cannabinoid CB₁ antagonistic activity of compounds of the formulas (I), (II), III), (IV) or (V) is also described in the WO 03/026647, WO 03/027076, WO 03/026648 or WO 03/078413, and was determined by functional studies using CHO cells in which human cannabinoid CB₁ receptors are stably expressed. Adenylyl cyclase was stimulated using forskolin and measured by quantifying the amount of accumulated cyclic AMP. Concomitant activation of CB₁ receptors by CB₁ receptor agonists (e.g. CP-55,940 or (R)-WIN-55,212-2) can attenuate the forskolin-induced accumulation of cAMP in a concentration-dependent manner. This CB₁ receptor-mediated response can be antagonized by CB₁ receptor antagonists such as the compounds used in the present invention.

The cannabinoid CB₁ receptor antagonistic, agonistic or partial agonistic activity of compounds e.g. formula (V) of the invention can be also determined by functional studies using the human CB₁ receptor cloned in Chinese hamster ovary (CHO) cells according to the following protocol. CHO cells were grown in a DMEM culture medium, supplemented with 10% heat-inactivated fetal calf serum. Medium was aspirated and replaced by DMEM, without fetal calf serum, but containing [³H]-Arachidonic acid and incubated overnight in a cell culture stove (5% CO₂/95% air; 37° C.; water-saturated atmosphere). During this period [³H]-Arachidonic acid was incorporated in membrane phospholipids. On the test day, medium was aspirated and cells were washed three times using 0.5 ml phosphate-buffered saline, containing 0.2% bovine serum albumin. Stimulation of the CB₁ receptor by WIN 55,212-2 led to activation of PLA₂ followed by release of [³H]-Arachidonic acid into the medium. This WIN 55,212-2-induced release was concentration-dependently antagonized by CB₁ receptor antagonists.

Cannabinoid receptor agonistic or partial agonistic activity of compounds of the invention can be determined according to published methods, such as assessment of in vivo cannabimimetic effects (Wiley, J. L. Jefferson et al., J. Pharmacol. Exp. Ther. 2001, 296, 1013).

Cannabinoid receptor antagonists may behave as inverse agonists (Landsman, R. S. et al., Eur. J. Pharmacol. 1997, 334, R1-R2).

The whole content of the international patent applications WO 03/026647, WO 03/027076, WO 03/026648 and WO 03/078413 is incorporated by reference into the present application regarding the disclosure of the CB₁ antagonistic compounds of the formulas (I), (II), III), (IV) or (V) used according to the present invention in combination with lipase inhibitors.

The outstanding unique pharmacological profile of compounds with CB₁-receptor activity and the formulas (I), (II), III), (IV) or (V) which are antagonists of the cannabis CB₁-receptor, as well as prodrugs, tautomers and salts thereof, includes particularly high safety and tolerability also in combination with other drugs, in particular in combination with lipase inhibiting compounds according to the present invention. Thus the CB₁ antagonistic compounds of the formulas (I), (II), III), (IV) or (V) in combination with lipase inhibiting compounds are particularly suitable also in patient groups with enhanced need of safety and tolerability, in particular such as juvenile patients and/or patients subject to long term treatment, e.g. in drug induced obesity.

This safety and tolerability of CB₁ antagonistic compounds of the formulas (I), (II), III), (IV) or (V) in combination with lipase inhibiting compounds is advantageous in the treatment and/or prophylaxis of obesity in those patient populations where a single treatment is not sufficiently effective and a combination treatment and/or prophylaxis involving different medical or metabolic mechanisms is desired or required for achieving and stabilizing a defined degree of weight loss.

Hence, combination of CB₁ antagonistic compounds of the formulas (I), (II), III), (IV) or (V) in combination with lipase inhibiting compounds according to the present invention is expected to be very is advantageous in the treatment and/or prophylaxis of obesity in general, e.g. of obesity in adolescent patients of any age, and particularly also in pediatric or juvenile obesity, and in drug induced obesity.

The CB₁ receptor modulating activity of the compounds of the formulas (I), (II), III), (IV) or (V) of the invention makes them particularly useful in the treatment of obesity, juvenile obesity and drug induced obesity, when used in combination with lipase inhibitors. Specific examples of lipase inhibiting compounds which can be used in such combination preparations are (but not restricted to) the synthetic lipase inhibitor orlistat, panclicins, lipase inhibitors isolated from micro organisms such as lipstatin (from Streptomyces toxytricini), ebelactone B (from Streptomyces aburaviensis), synthetic derivatives of these compounds, 2-oxy-4H-3,1-benzoxazin-4-one derivatives like ATL-962 and structurally related compounds, 2-amino-4H-3,1-benzoxazin-4-one derivatives, as well as extracts of plants known to possess lipase inhibitory activity, for instance extracts of Alpinia officinarum Hance or compounds isolated from such extracts like 3-methylethergalangin (from A. officinarum). The lipase inhibiting compound may also be a lipase inhibiting polymer. These lipase inhibiting compounds and their manufacture are well known in the state of the art.

Lipase inhibiting compounds used in the combinations according to the present invention may be any lipase inhibiting compound suitable for pharmaceutical use, e.g. in particular inhibitors of pancreatic lipases. Lipases are key enzymes in the digestive system which break down tri- and diglycerides, which are too large to be absorbed by the small intestine into fatty acids which can be absorbed. Since lipases are responsible for the hydrolysis of fat, a consequence of their inhibition is a reduction in fat hydrolysis and absorption. Therefore, inhibition of lipases results in a reduction in the absorption of fat. The lipase inhibiting compound is preferably the synthetic lipase inhibitor orlistat and structurally related compounds, 2-oxy-4H-3,1-benzoxazin-4-one derivatives like ATL-962 and structurally related compounds, 2-amino-4H-3,1-benzoxazin-4-one derivatives, lipase inhibitors isolated from micro organisms such as lipstatin, ebelactone B, or synthetic derivatives of these compounds, however may also be a lipase inhibiting polymer. Most preferred are orlistat, panclicins, ATL-962 and lipstatin.

Orlistat (tetrahydrolipstatin) and lipstatin are described in the U.S. Pat. No. 4,598,089 and its European equivalent EP 0 129 748 B1 in more detail. The compounds are 2-hexyl-3-hydroxy-hexadecanoic acid lactone derivatives with the chemical names (2S,3S,5S,7Z,10Z)-5-((S)-2-formamido-4-methylvaleryloxy)-2-hexyl-3-hydroxy-7,10-hexadecadienoic acid lactone (lipstatin) and (2S,3S,5S)-5-((S)-2-4-methylvaleryloxy)-2-heyl-3-hydroxy-hexadecanoic acid lactone (tetrahydrolipstatin). The compounds are known to be inhibitors of pancreas lipase which can be used for the prevention of treatment of obesity and hyperlipaemia, for which purpose they can be formulated as medicaments or incorporated into industrially prepared foodstuffs. Inhibition of pancreas lipase prevents the hydrolysis of dietary fats to give absorbable free fatty acids and monoglycerides, so that the fats are excreted unchanged. IC50's for lipstatin and tetrahydrolipstatin for inhibition of hydrolysis of triolein by porcine pancrease lipase are 0.07 and 0.18 mcg/ml, respectively.

Furthermore, there are suitable lipase inhibitors which are structurally related to orlistat and/or lipstatin and which are known as panclicins. These panclicines are derived from orlistat and contain a 4-ring lactone (Mutoh M; Nakada N; Matsukima S; Ohshima S; Yoshinari K; Watanabe J Location: Kanagawa, Japan Issue Date: 19-JAN-1995 Journal: J. Antibiot., 47, No. 12, 1369-75, 1994). The biological data of these panclicins may be summarized as follows: Panclicins A, B, C, D and E, structural analogs of tetrahydrolipstatin (THL), dose-dependently inhibited hydrolysis of triolein of fatty acids by porcine pancreatic lipase, with IC50 values of 2.9, 2.6, 0.62, 0.66 and 0.89 microM, respectively. The inhibitory activity of panclicins A and B (alanine moiety in place of leucine in THL) was 2-3-fold weaker than that of THL; in contrast, the inhibitory activity of panclicins C, D and E (glycine moiety in place of leucine in THL) was 2-fold stronger than that of THL. Panclicins A, B, C, D and E also potently inhibited plasma lipases with IC50 values of 1.0, 1.2, 0.29, 0.25 and 0.15 microM, respectively. Panclicins A and B inhibited plasma lipases with the same potency as THL, while panclicins C, D and E had a 3-6-fold greater inhibitory activity than THL. Panclicins A, B, C, D and E inhibited bacterial and fungal lipases with profiles similar to those for porcine pancreatic lipase. Panclicins inhibited pancreatic lipase irreversibly, but less irreversibly than THL. Panclicins A, B, C, D and E irreversibly inhibit pancreatic lipase.

Ebelactone B is described in the U.S. Pat. No. 4,358,602 and its German equivalent DE 3 109 335 C1. Ebelactone A and ebelactone B belong to a group of compounds that exhibit activity to enhance the cell mediated immune response in living animals and they also inhibit inflammations in living animals. Thus they may be used in the immunological treatment of tumours and for enhancing anti-tumour agents such as bleomycins. The compounds have anti-esterase activity and anti-formylmethionine aminopeptidase activity. Administration to mice of these compounds at a dosage of 0.781-50 mg/kg (i.p.) or 0.5 mg/kg (per os) enhances the development of DTH response and the compounds show a potentiating effect on cell-mediated immunity. Ebelactone B reduces carragheenin-induced swelling in mice.

In the context of the present invention the lipase inhibitors administered in combination with the CB₁ antagonistic compounds to a patient for treating obesity may be also a polymer that has been substituted with or comprises one or more groups which can inhibit a lipase. Such lipase inhibiting polymers are described in the U.S. Pat. No. 6,572,850, U.S. Pat. No. 6,558,657, U.S. Pat. No. 6,352,692, U.S. Pat. No. 6,267,952 and in the international patent application WO 99/34786. In one embodiment, the lipase inhibiting group can be a “suicide substrate” which inhibits the activity of the lipase by forming a covalent bond with the enzyme either at the active site or elsewhere. In another embodiment, the lipase inhibiting group is an isosteric inhibitor of the enzyme.

In a first aspect of the present invention when using lipase inhibiting polymers in addition to the CB₁ antagonistic compounds, the lipase inhibiting group inactivates a lipase such as gastric, pancreatic and lingual lipases. Inactivation can result by forming a covalent bond such that the enzyme is inactive. The covalent bond can be formed with an amino acid residue at or near the active site of the enzyme, or at a residue which is distant from the active site provided that the formation of the covalent bond results in inhibition of the enzyme activity. Lipases contain a catalytic triad which is responsible for the hydrolysis of lipids into fatty acids. The catalytic triad consists of a serine, aspartate and histidine amino acid residues. This triad is also responsible for the hydrolysis of amide bonds in serine proteases, and it is expected that compounds that are serine protease inhibitors will also inhibit lipases. Therefore, serine protease inhibitors that can be covalently linked to a polymer are preferred lipase inhibiting groups. For example, a covalent bond can be formed between the lipase inhibiting group and a hydroxyl at or the catalytic site of the enzyme. For instance, a covalent bond can be formed with serine. Inactivation can also result from a lipase inhibiting group forming a covalent bond with an amino acid, for example cysteine, which is at some distance from the active site. In a second aspect of the present invention when using lipase inhibiting polymers in addition to the CB₁ antagonistic compounds, non-covalent interaction between the lipase inhibiting group and the enzyme can also result in inactivation of the enzyme. For example, the lipase inhibiting group can be an isostere of a fatty acid, which can interact non-covalently with the catalytic site of the lipase. In addition, the lipase inhibiting group can compete for lipase hydrolysis with natural triglycerides.

A variety of polymers can be employed in the invention described herein. The polymers can be aliphatic, alicyclic or aromatic or synthetic or naturally occurring. However, aliphatic and alicyclic synthetic polymers are preferred. Furthermore, the polymer can be hydrophobic, hydrophilic or copolymers of hydrophobic and/or hydrophilic monomers. The polymer can be non-ionic (e.g., neutral), anionic or cationic, in whole or in part. Furthermore, the polymers can be manufactured from olefinic or ethylenic monomers (such as vinylalcohol) or condensation polymers. For example, the polymers can be a polyvinylalcohol, polyvinylamine, poly-N-alkylvinylamine, polyallylamine, poly-N-alkylallylamine, polyalkylenimine, polyethylene, polypropylene, polyether, polyethylene oxide, polyamide, polyacrylic acid, polyalkylacrylate, polyacrylamide, polymethacrylic acid, polyalkylmethacrylate, polymethacrylamide, poly-N-alkylacrylamide, poly-N-alkylmethacrylamide, polystyrene, vinyinaphthalene, ethylvinylbenzene, aminostyrene, vinylbiphenyl, vinylanisole, vinylimidazolyl, vinylpyridinyl, dimethylaminomethylstyrene, trimethylammoniumethylmethacrylate, trimethylammoniumethylacrylate, carbohydrate, protein and substituted derivatives of the above (e.g., fluorinated monomers thereof) and copolymers thereof. Preferred polymers include polyethers, such as polyalkylene glycols.

The polymers employed in the methods described herein as well as intermediates and methods for preparing the polymers are described in detail in the U.S. Pat. No. 6,572,850, U.S. Pat. No. 6,558,657, U.S. Pat. No. 6,352,692, U.S. Pat. No. 6,267,952 and in the international patent application WO 99/34786, which are all incorporated by reference into the present invention.

Recently, in the international patent application WO 03/072555 new 5-hydrocarbyloxy-3-phenyl-1,3,4-oxadiazol-2-ones of formula (A) are described to be pancreatic lipase inhibitors useful for treating metabolic diseases, cardiovascular diseases or especially obesity.

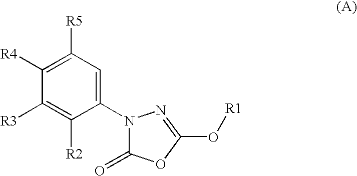

Such oxadiazolones of formula (A) and their salts and acid addition salts are also suitable for combinations with the CB₁ antagonistic compounds used according to the present invention. In formula (A) the substituents may be as follows:
R1 may be 7-22C alkyl; 24C alkyl substituted by 4-20C alkoxy, 6-1° C. aryl, 6-10C aryloxy or (4-12C) alkoxy-(2-4C) alkoxy (where aryl can be substituted by one or more of halogen, 1-4C alkyl, 1-4C alkoxy, NO2 or CF3); 7-20C alkenyl; or phenyl substituted by 6-12C alkyl or by phenoxy; and
R2 to R5 each may be H, halogen, NO2, 1-4C alkyl, 1-4C alkoxy, CF3 or OCF3; or (6-10C) aryl-(14C) alkoxy, 6-10C aryloxy, 6-10C aryl, 3-8C cycloalkyl or 3-8C cycloalkoxy (optionally substituted by halogen, CF3, 1-4C alkoxy or 1-4C alkyl).

These 5-hydrocarbyloxy-3-phenyl-1,3,4-oxadiazol-2-ones are described to have pharmacological properties as anorectic, antidiabetic, hypotensive or cardiant, with mechanism of action as pancreatic lipase inhibitors. For example 5-dodecyloxy-3-(4-trifluoromethoxy-phenyl)-3H-(1,3,4)-oxadiazol-2-one had IC50 0.03 microM for inhibition of porcine pancreatic lipase. Hence, these 5-hydrocarbyloxy-3-phenyl-1,3,4-oxadiazol-2-ones may be used as medicaments, especially for the treatment of obesity. As pancreatic lipase inhibitors, the 5-hydrocarbyloxy-3-phenyl-1,3,4-oxadiazol-2-ones inhibit the resorption of the fat content of foods and thus reduce fat uptake and body weight (or prevent increase in body weight). Furthermore, the 5-hydrocarbyloxy-3-phenyl-1,3,4-oxadiazol-2-ones are reported to also have a beneficial effect in the treatment of metabolic disorders (e.g. diabetes) or cardiovascular disorders (e.g. hypertension and cardiac infarction). The lipase inhibiting compounds of formula (A) are described in more detail in the WO 03/072555 and can be obtained according to known methods. A suitable synthesis for the lipase inhibiting compounds of formula (A) is described also in the international patent application WO 03/072555. The whole content of the international patent application WO 03/072555 is incorporated by reference into the present application regarding the disclosure of lipase inhibitors of formula (A).

In addition, in the international patent application WO 03/072098 further 5-hydrocarbyloxy-3-phenyl-1,3,4-oxadiazol-2-ones of formula (A) are described to be pancreatic lipase inhibitors useful for treating of obesity or diabetes mellitus type 1 and 2. Such oxadiazolones of formula (A) as described in WO 03/072098 and their salts and acid addition salts are also suitable for combinations with the CB₁ antagonistic compounds used according to the present invention. In formula (A) the substituents may be as follows:
R1 may be 1-6C alkyl; 3-9C-cycloalkyl, both groups optionally may be substituted by phenyl, 1-4C alkoxy, S-1-4C alkyl, N(1-4C-alkyl)2; and phenyl optionally may be also substituted by halogen, 1-4C alky, 1-4C-alkyloxy, nitro or CF3; and
R2 to R5 each may be independently H, halogen, NO2, 1-4C alkyl, 1-9C alkoxy which is substituted by F, 6-10C-aryl, amino or 1-4C alkyl amino;
   6-10C-aryl-1-4C-alkyloxy, 6-10C-aryloxy, 6-10C-aryl, 6-10C-aryloxy-1-4C-alkyl, 3-8C cycloalkyl or O(3-8 cycloalky), which may optionally be substituted by halogen, CF3, 1-4 alkyloxy or 1-4C alkyl;
   SO2-NH-(1-6C alkyl), optionally substituted by N(1-6C alkyl)2, SO2—NH-(2,2,6,6-tetramethylpiperidin-4-yl), SO2-NH-(3-8C cycloalkyl), optionally substituted by 1-4C alkyl, SO2-N(1-6Calkyl)2 or COX;
   2-oxo-pyrrolidin-1-yl, 2,5-dimethylpyrrol-1-yl or NR6-A-R7
   with the proviso that R2, R3, R4 and R5 are not H at the same time when
   X is O(1-6C alkyl), NH(1-6C alkyl), NH(3-8C cycloalkyl or N(1-6C alkyl)2 and N(1-6C alkyl)2 may be also pyrrolidino, piperidino, morpholino, thiomorpholino or piperazino, which optionally substituted by 1-4C alkyl, benzyl, 6-10C aryl, CO-(1-4 alkyl), CO-(6-10 aryl), CO—O-(1-4C alkyl), SO2-(1-4C alkyl) or SO2-(6-10C aryl);
   R6 is H, 1-4C alkyl or 6-10C-aryl-14C-alkyl, wherein aryl may be substituted by halogen, CF3, 1-8C alkyloxy or 1-4C alkyl;
   A is a single bond, COn, SOn or CONH;
   n is 1 oder 2:
   R7 is H; 1-18C alkyl or 2-18C alkenyl, which may up to three times substituted by 1-4C alkyl, halogen, CF3, 1-4C alkyloxy, N(1-4C alkyl)2, —COOH, 1-4C alkyloxycarbonyl, 6-12C aryl, 6-12C aryloxy, 6-12C arylcarbonyl, 6-10-aryl-1-4C-alkyloxy or oxo, wherein aryl itself may be optionally substituted by halogen, 1-4C alkyl, aminosulfonyl or methylmercapto;
      6-10C-aryl-1-4C-alkyl, 5-8C-cycloalkyl-1-4C-alkyl, 5-8C cycloalkyl, 6-10-aryl-2-6C-alkenyl, 6-10C aryl, biphenylyl, diphenyl-(14 alkyl), indanyl, which may be optionally substituted by 1-18C alkyl, 1-18C alkyloxy, 3-8C cycloalkyl, COOH, hydroxy, 14C alkylcarbonyl, 6-10C-aryl-1-4C alkyl, 6-10C-aryl-1-4C-alkyloxy, 6-10C aryloxy, nitro, cyano, 6-10C aryl, fluorsulfonyl, 1-6C alkyloxycarbonyl, 6-10 arylsulfonyloxy, pyridyl, NHSO2-(6-10 aryl), halogen, CF3 or OCF3, wherein alkyl may be additionally substituted by 14C alkyloxycarbonyl, CF3 or carboxy and aryl by halogen, CF3 or 1-4C alkyloxy;
      or the group Het-(CH2)r- with r=0, 1, 2 or 3 and Het=saturated or unsaturated 5-7-membered heterocyclus, which may be optionally benzo anellated and substituted by 1-4C alkyl, 6-10C aryl, halogen, 1-4C alkyloxy, 1-4C alkyloxycarbonyl, 6-10C-aryl-1-4Calkyl, 6-10C-aryl-1-4C-alkylmercapto or nitro, wherein benzo anellated aryl may be substituted by halogen, 1-4C alkyloxy or CF3 and alkyl in arylalkyl may be substituted by methoxy and CF3.

The lipase inhibiting compounds of formula (A) are described in more detail in the WO 03/072098 and can be obtained according to known methods. A suitable synthesis for the lipase inhibiting compounds of formula (A) is described also in the international patent application WO 03/072098. The whole content of the international patent application WO 03/072098 is incorporated by reference into the present application regarding the disclosure of lipase inhibitors of formula (A).

In addition, in the U.S. Pat. No. 6,624,161 and its corresponding international patent application WO 00/040,569 and WO 00/40247 further lipase inhibiting compounds are described which are also suitable in the context of the present invention for combination with CB1 antagonistic compounds described herein. These patent documents U.S. Pat. No. 6,624,161 and WO 00/040,569 describe a series of compounds which are 2-oxy-4H-3,1-benzoxazin-4-one derivatives, including ATL-962, and their use in obesity and obesity-related disorders, including type 2 diabetes. The 2-oxy-4H-3,1-benzoxazin-4-one derivatives have the formula (B) or are or a pharmaceutically acceptable salt, ester, amide or prodrug thereof: 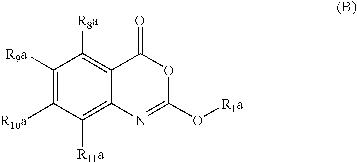
wherein:
R1a is
   (i) a C10-30 branched or unbranched alkyl, optionally substituted by one or more independently of C3-6 cycloalkyl, C3-6 cycloalkenyl, aryl, heteroaryl, reduced heteroaryl, —C(O)R13, —CO2R13, —SOR13, —SO2R13, —NR13R14, —OR13, —SR13, —C(O)NR13R14, —NR14C(O)R13, halogen, cyano, and nitro and/or optionally interrupted by one or more oxygen atoms with the proviso that any hetero atom in R1a must be separated from the exocyclic oxygen atom (or from any other heteroatom) by at least two carbon atoms;
   (ii) C2-25 alkenyl, C2-25 alkynyl, C3-6 cycloalkenyl, aryl-C2-25 alkenyl, heteroaryl-C2-25 alkenyl, reduced heteroaryl, reduced heteroaryl-C1-25 alkyl or a substituted derivative of any of the foregoing groups wherein the substituents are one or more independently of C1-6 alkyl, halosubstituted C1-6 alkyl, aryl, aryl-C1-6 alkyl, heteroaryl, reduced heteroaryl, reduced heteroaryl-C1-6 alkyl, C1-6 alkoxy, aryl-C1-6 alkoxy, —C(O)R13, —CO2R13, —SOR13, —SO2R13, —NR13R14, —OR13, —SR13, —C(O)NR13R14, —NR14C(O)R13, halogen, cyano, and nitro, with the proviso that any hetero atom in R1a must be separated from the exocyclic oxygen atom (or from any other heteroatom) by at least two carbon atoms;
   (iii) a C2-9 alkyl group interrupted by one or more oxygen atoms and optionally substituted by one or more independently of C3-6 cycloalkyl, C3-6 cycloalkenyl, aryl, heteroaryl, reduced heteroaryl, —C(O)R13, —CO2R13, —SOR13, —SO2R13, NR13R14, OR13, SR13, —C(O)NR13R14, —NR14C(O)R13, halogen, cyano and nitro with the proviso that any hetero atom in R1a must be separated from the exocyclic oxygen atom (or from any other heteroatom) by at least two carbon atoms; or
   (iv) a C1-9 alkyl group substituted by a group selected from —C(O)R13, —CO2R13, SOR13, SO2R13, NR13R14, OR13, SR13, C(O)NR13R14, NR14C(O)R13; tetrahydronaphthyl, pyridyl, pyrrolyl, piperidinyl, halogen, cyano, nitro, bicyclic aryl, bicyclic heteroaryl, monocyclic or bicyclic reduced heteroaryl, monocyclic heteroaryl other than imidazolyl;
   (v) a phenyl group substituted by a group selected from OR17, —COR13, —CO2R13, SOR13, SO2R13, CONR13R14, NR14C(O)R13; halosubstituted C1-6 alkyl, aryl, arylC1-6 alkyl, heteroaryl and heteroarylC1-6 alkyl; or
   (vi) a bicyclic aryl, bicyclic heteroaryl, monocyclic or bicyclic reduced heteroaryl, or monocyclic heteroaryl group other than imidazolyl, optionally substituted by a group selected from OR17, —COR13, —CO2R13, SOR13, SO2R13, CONR13R14, NR14 C(O)R13; halosubstituted C1-6 alkyl, aryl, arylC1-6 alkyl, heteroaryl and heteroarylC1-6 alkyl;
      where R13 and R14 each independently represents hydrogen, C1-10 alkyl, C2-10 alkenyl, C2-10 alkynyl, C3-6 cycloalkyl, C3-6 cycloalkenyl, aryl, arylC1-10 alkyl, heteroaryl, heteroarylC1-10 alkyl, reduced heteroaryl or reduced heteroaryl, C1-10 alkyl, and R17 represents hydrogen or C2-10 alkenyl, C2-10 alkynyl, C3-6 cycloalkyl, C3-6 cycloalkenyl, aryl, arylC1-10 alkyl, heteroaryl, heteroarylC1-10 alkyl, reduced heteroaryl or reduced heteroarylC1-10 alkyl
and R8a, R9a, R10a and R11a are each independently hydrogen, halo, hydroxy, amino, nitro, cyano, thiol, C1-10 alkyl, C1-10 alkoxy, C1-10 cycloalkyl, C1-10 cycloalkoxy, C(O)R15, C(O)NR15R16, S(O)R15 or haloC1-10 alkyl;
where R15 and R16 each independently represent hydrogen or C1-10 alkyl with the proviso that when R8a, R9a, R10a, and R11a are H, R1a is not CH2CH2Cl or C3 alkenyl.

Furthermore in the international patent application WO 00/40247 related 2-amino-4H-3,1-benzoxazin-4-one derivatives are described as lipase inhibiting compounds for the treatment of obesity. In formula (B) then the —OR1a substituent is replaced by a —NR1R2 group with the definitions for R1 and R2 as given in the WO 00/40247.

The above group of structurally related compounds include ATL-962, an oral non-absorbed synthetic lipase inhibitor derived from Alizyme's pancreatic lipase inhibitor research program, is under development for the potential treatment of obesity and the potential management of type 2 diabetes. ATL-962 has the chemical name 2-hexadecyloxy-6-methyl-4H-3,1-benzoxazin-4-one. Preclinical studies showed that ATL-962 had similar efficacy to orlistat and no toxicity was observed. Clinical data for these compounds is also available in the public domain, e.g. resulting from clinical studies with ATL-962 in obesity.

Thus, the results from a phase Ib program with ATL-962 were presented at the International Congress of Obesity in Sao Paulo, Brazil. The three phase Ib trials involved a total of 99 healthy male volunteers in groups of seven or nine, given one of several doses of ATL-962 (66 subjects) or placebo (24 subjects), tid (three times in day) with food for 5 days. In one group the nine subjects were given orlistat (qv) 120 mg tid. Overall, ATL-962 was safe and well tolerated and showed evidence of efficacy as indicated by an increase in excretion of fat from the diet. Subjects given doses between 50 mg and 300 mg ATL-962 bid with meals excreted fat at an average of between 4.9 (+/4.3) and 11.2 (+/−6.9) g/day compared to 1.4 (+/−1.0) g/day on placebo and 5.6 (+1-3.8) g/day on orlistat. Compared to placebo, 55% of subjects who received ATL-962 (50 mg to 300 mg) demonstrated a 3-fold or greater increase in fat excretion and 27% of subjects demonstrated a 7-fold or greater increase. There was evidence of dose-dependency. Adverse events and their frequency were similar between ATL-962 and placebo and were mainly gastrointestinal, with the predominant event being oily stool.

The results of a multicenter, randomized, double-blind, parallel-group trial (phase IIb study), involving 370 clinically obese patients, was being performed in specialist clinics in 5 European countries, and in September 2003 preliminary results were reported. All dose levels of ATL-962 (60, 120 and 240 mg) demonstrated a significant reduction in weight, compared to placebo, for all treatment groups. There was no difference in the extent of weight loss between treatment groups. LDL-cholesterol decreased in the treatment groups, but not for placebo. There was no difference in HDL-cholesterol levels in the treatment groups, whilst it increased in placebo-treated patients. Total cholesterol decreased in the treatment groups, whilst placebo showed an increase. ATL-962 was safe and generally well tolerated.

The lipase inhibiting compounds of formula (B) like ATL-962 and structurally related compounds are described in more detail in the U.S. Pat. No. 6,624,161 and its corresponding international patent application WO 00/040,569, and can be obtained according to known methods. A suitable synthesis for the lipase inhibiting compounds of formula (B) is described also in the U.S. Pat. No. 6,624,161 and international patent application WO 00/040,569. The whole content of the U.S. Pat. No. 6,624,161 and international patent application WO 00/040,569 is incorporated by reference into the present application regarding the disclosure of lipase inhibitors of formula (B). The whole content of the international patent application WO 00/040,247 is also incorporated by reference into the present application regarding the disclosure of lipase inhibitors described therein, with related 2-amino-4H-3,1-benzoxazin-4-one compound structure.

Pharmaceutically acceptable salts, hydrates and solvates, and prodrugs of all the above described lipase inhibiting compounds may also be used in the context of the present invention.

The CB₁ antagonistic compound of the formulas (I), (II), III), (IV) or (V), or a prodrug, tautomer or salt thereof, and the lipase inhibiting compound used according to the invention can be brought into forms suitable for pediatric treatment and/or prophylaxis of the diseases indicated above, and in particular of obesity, e.g. for adolescent or pediatric administration, as well as for the administration in treating drug induced obesity by means of usual processes using pharmaceutical excipients, auxiliary substances and/or liquid or solid carrier materials. As therapeutic agents, the CB₁ antagonistic compound and/or the lipase inhibiting compounds may be contained together with (conventional) pharmaceutical excipients, adjuvants and/or auxiliaries in pharmaceutical preparations such as tablets, capsules, suppositories or solutions. These pharmaceutical preparations may be prepared according to known methods, using conventional solid or liquid vehicles such as lactose, starch or talc, or liquid paraffins and/or using (conventional) pharmaceutical excipients, adjuvants and/or auxiliaries, such as tablet disintegrating agents, solubilisers or preservatives.

Hence, in a further aspect the invention also pertains to a pharmaceutical composition containing at least one compound with CB₁-receptor activity of the formulas (I), (II), III), (IV) or (V), or a prodrug, tautomer or salt thereof, in combination with at least one lipase inhibiting compound. A preferred pharmaceutical composition contains at least one compound of the formulas (I), (II), III), (IV) or (V) as defined above in combination with at least one lipase inhibiting compound as combined active components. A further pharmaceutical composition according to the invention contains as active components at least one compound with CB₁-receptor activity having the formula (I), (II), III), (IV) or (V) as defined above, preferably the CB1 antagonistic compound, or a prodrug, tautomer or salt thereof, and at least one lipase inhibiting compound for the treatment and/or prophylaxis of obesity in adolescent or in juvenile patients and/or for the treatment and/or prophylaxis of drug induced obesity in juvenile as well as adolescent patients. Particular pharmaceutical compositions according to the invention, are characterized in that the at least one compound with CB₁-receptor activity having the formula (I), (II), III), (IV) or (V) as defined above, preferably the CB₁ antagonist, or the prodrug, tautomer or salt thereof, and the at least one lipase inhibiting compound each are present in an amount effectively suited for the treatment and/or prophylaxis of obesity in a juvenile patient in need of such treating. In a further embodiment of the invention the CB₁ antagonistic compound, in particular the CB₁ antagonistic compound of the formulas (I), (II), III), (IV) or (V), and the lipase inhibiting compound are each present in the pharmaceutical composition in an amount effectively suited for the treatment and/or prophylaxis of drug induced obesity in juvenile as well as adolescent patients in need of such treating. In the pharmaceutical compositions according to the invention the CB₁ antagonistic compound, preferably the CB₁ antagonistic having the formula (I), (II), III), (IV) or (V), or the prodrug, tautomer or salt thereof, is used preferably in combination with at least one lipase inhibiting compound selected from the group of lipase inhibiting polymers, orlistat, panclicins, ATL-962 and lipstatin.

The invention also pertains to a pharmaceutical product containing as a medicament a CB₁ antagonistic compound, preferably the CB₁ antagonistic compound having one of the formulas (I), (II), III), (IV) or (V) as defined above, or a prodrug, tautomer or salt thereof, and a leaflet indicating that said CB₁ antagonistic compound may be administered in combination with a lipase inhibiting compound for simultaneous, separate or step-wise administration in the treatment and/or prophylaxis of obesity.

Finally the invention also includes a method of treatment and/or prophylaxis of obesity, e.g. in adolescent or in juvenile patients and/or for the treatment and/or prophylaxis of drug induced obesity in juvenile as well as adolescent patients, characterized in that a CB₁ antagonistic compound, in particular a compound of the formula (I), (II), III), (IV) or (V), which is an antagonist of the cannabis CB₁-receptor, or a prodrug, tautomer or salt thereof, is administered in combination with at least one lipase inhibiting compound to a patient in need of such treating. In a preferred method of treatment and/or prophylaxis of obesity according to the invention a CB₁ antagonistic compound which is a compound of one of the formulas (I), (II), III), (IV) or (V) as defined above, or a prodrug, tautomer or salt thereof, is administered in combination with at least one lipase inhibiting compound. The method of treatment and/or prophylaxis of obesity according to the present invention may be directed to obesity in adolescent or in juvenile patients and/or to drug induced obesity in juvenile as well as adolescent patients. In a variant of the invention the method of treatment and/or prophylaxis is characterized in that the treating is directed to obesity in juvenile patients. In a further variant of the invention the method of treatment and/or prophylaxis is characterized in that the treating is directed to drug induced obesity in juvenile or adolescent patients. In the method of treatment and/or prophylaxis according to the invention the CB₁ antagonistic compound, preferably the CB₁ antagonistic compound having one of the formulas (I), (II), III), (IV) or (V) as defined above, or a prodrug, tautomer or salt thereof, is administered preferably in combination with at least one lipase inhibiting compound selected from the group of lipase inhibiting polymers, orlistat, panclicins, ATL-962 and lipstatin.

According to the invention the CB₁ antagonistic compound, preferably the CB1 antagonistic compound having one of the formulas (I), (II), III), (IV) or (V) as defined above, or a prodrug, tautomer or salt thereof, is administered in combination with the lipase inhibiting compound by simultaneous, separate or step-wise administration route.

The compounds used in the combinations or compositions according to the present invention each are preferably administered to a patient in need thereof and in a quantity sufficient to prevent and/or treat the symptoms of the condition, disorder or disease, e.g. obesity. For all aspects of the invention, particularly medical ones, the administration of a compound or composition has a dosage regime which will ultimately be determined by the attending physician and will take into consideration such factors such as the compound being used, animal type, age, weight, severity of symptoms, method of administration, adverse reactions and/or other contraindications. Specific defined dosage ranges can be determined by standard design clinical trials with patient progress and recovery being fully monitored. Such trials may use an escalating dose design using a low percentage of the maximum tolerated dose in animals as the starting dose in man.

The physiologically acceptable compounds used in the combinations or compositions according to the present invention each are will normally be administered in a daily dosage regimen (for an adult patient) of, for example, an oral dose of between 1 mg and 2000 mg, preferably between 30 mg and 1000 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a physiologically acceptable salt thereof calculated as the free base, the compound being administered 1 to 4 times per day. Suitably the compounds will be administered for a period of continuous therapy, for example for a week or more. For a juvenile patient usually a part of the oral dose for an adult patient is administered, e.g. 1 fifth to 1 half of the oral dose described before for an adult patient.

Preferably, in one embodiment of the invention the method of treatment and/or prophylaxis is directed to the treating of obesity in juvenile patients. In another preferred embodiment of the invention the method of treatment and/or prophylaxis is directed to the treating of drug induced obesity in juvenile or adolescent patients. This drug induced obesity may be in particular caused by drugs like atypical antipsychotics.

In one embodiment of the invention the method of treatment and/or prophylaxis is directed to the treating of obesity in juvenile patients. Thus, it is advantageous that Cannabinoid antagonists in combination with lipase inhibitors are particularly suitable for the treatment of Childhood Obesity and related Comorbidities as for example Type 2 Diabetes. There is a clear medical need for improved therapy as obesity has become an increasingly important medical problem not only in the adult population but increasingly in children and (young and older) adolescents. In national surveys from the 1960s to the 1990s in the United States, the prevalence of overweight in children grew from 5% to 11% (Sorof and Daniels 2002). In Canada as another example childhood obesity has tripled in the past 20 years (Spurgeon 2002). Obesity in childhood causes a wide range of serious complications, and increases the risk of premature illness and death later in life, raising public-health concerns (Ebbeling, Pawlak et al. 2002). Over the last decades a tremendous increase of cases of type 2 diabetes was observed, especially also in children. This epidemic trend is clearly reflecting the increasing rates of obesity. Type-2-diabetes was in the past considered a disease of adults and older individuals, not a pediatric condition (Arslanian 2002). One of the main risk factor of pediatric type 2 diabetes is obesity.

Type 2 diabetes in children (as is in adults) is part of the insulin resistance syndrome (Rosenbloom 2002) that includes hypertension, dyslipidemia and other atherosclerosis risk factors, and hyperandrogenism seen as premature adrenarche and polycystic ovary syndrome. Other outcomes related to childhood obesity include left ventricular hypertrophy, nonalcoholic steatohepatitis, obstructive sleep apnea, orthopedic problems, and severe psychosocial problems.

In addition primary hypertension has become increasingly common in children again associated obesity as a major independent risk factor. Obese children are at approximately a 3-fold higher risk for hypertension than non-obese children (Sorof and Daniels 2002). The benefits of weight loss for blood pressure reduction in children have been demonstrated in both observational and interventional studies.

Public concerns are rising because of a rapid development of the childhood obesity epidemic in genetically stable populations. Driving factors are assumed to be mainly adverse environmental factors for which straightforward recommendations of life style modifications exists. Obesity and it's related co-morbidities are very serious medical conditions and state of the art measures and treatment of obesity and especially childhood obesity remain largely ineffective at the time being (Ebbeling, Pawlak et al. 2002). The management of type 2 diabetes in is also especially difficult in children and the adolescent age group (Silink 2002). Craving for and over consumption of palatable food is one of the important factors of life-style related obesity in humans and especially also in children and adolescents. Treatment of type 2 diabetes and other co-morbid conditions by the degree of metabolic derangement and symptoms: The only data on the use of oral hypoglycemic agents in children with type 2 diabetes has been with metformin (Rosenbloom 2002).

Thus, CB₁ antagonists used according to the present invention in combination with lipase inhibitors offer a unique opportunity for the treatment of obesity by interacting with these “driving forces”. They are superior to current medical treatments and especially suited for adolescent as well as pediatric treatment because of their outstanding safety profile and/or tolerability and surprisingly beneficial combination effects. Besides efficacy, the treatment of obesity, especially the treatment of childhood obesity, dictated by safety.

Obesity in childhood is a medical condition that is likely to require long-term management. The safety profile of CB₁ antagonists used according to the present invention in combination with lipase inhibitors are suggested to be superior to current standard medications, and these CB₁ antagonists in combination with lipase inhibitors will be especially suited for the treatment and prevention of obesity in adolescents and in childhood obesity and related co-morbidities.

Literature:

Arslanian, S. (2002). “Type 2 diabetes in children: clinical aspects and risk factors.” Horm Res 57 Suppl 1: 19-28.
Ebbeling, C. B., D. B. Pawlak, et al. (2002). “Childhood obesity: public-health crisis, common sense cure.” Lancet 360(9331): 473-82.
Rosenbloom, A. L. (2002). “Increasing incidence of type 2 diabetes in children and adolescents: treatment considerations.” Paediatr Drugs 4(4): 209-21.
Silink, M. (2002). “Childhood diabetes: a global perspective.” Horm Res 57 Suppl 1:1-5.
Sorof, J. and S. Daniels (2002). “Obesity hypertension in children: a problem of epidemic proportions.” Hypertension 40(4): 441-7.
Spurgeon, D. (2002). “Childhood obesity in Canada has tripled in past 20 years.” Bmj 324(7351): 1416.

In another embodiment of the invention the method of treatment and/or prophylaxis is directed to the treating of drug induced obesity in juvenile or adolescent patients. Drug induced weight gain is also of major concern and subject to high medical need of improved treatments. Again, in this context the CB₁ antagonists in combination with lipase inhibitors according to the present invention are suggested to be superior to current standard medications, and these CB₁ antagonists in combination with lipase inhibitors will be especially suited for the treatment and prevention of drug induced obesity in juvenile as well as in adolescent patients.

Regarding drug induced weight gain, it is reported by Zimmermann, U., T. Kraus, et al. (2003, “Epidemiology, implications and mechanisms underlying drug-induced weight gain in psychiatric patients.” J Psychiatr Res 37(3): 193-220) that body weight gain frequently occurs during drug treatment of psychiatric disorders and is often accompanied by increased appetite or food craving. While occurrence and time course of this side effect are difficult to predict, it ultimately results in obesity and the morbidity associated therewith in a substantial part of patients, often causing them to discontinue treatment even if it is effective. Weight gain appears to be most prominent in patients treated with some of the second generation antipsychotic drugs and with some mood stabilizers. Marked weight gain also frequently occurs during treatment with most tricyclic antidepressants.

Very large weight gains are associated with drugs like for example the atypical antipsychotics clozapine and olanzapine. Some atypical antipsychotics, however, tend to cause significant weight gain, which may lead to poor compliance and other adverse health effects (Nasrallah, H. (2003). “A review of the effect of atypical antipsychotics on weight.” Psychoneuroendocrinology 28 Suppl 1: 83-96.). The mechanisms involved in antipsychotic drug-related weight gain are as yet uncertain, although serotoninergic, histaminic, and adrenergic affinities have been implicated along with other metabolic mechanisms. The atypical antipsychotics vary in their propensity to cause weight change with long-term treatment. Follow-up studies show that the largest weight gains are associated with clozapine and olanzapine, and the smallest with quetiapine and ziprasidone. Risperidone is associated with modest weight changes that are not dose related. Given the equivalent efficacy of atypical antipsychotics, weight-gain profile is a legitimate factor to consider when constructing an algorithm for treatment due to the serious medical consequences of obesity. In this regard co-administration of a CB₁ antagonist in combination with lipase inhibitors according to the invention is suggested to work beneficially.

### Experimental Protocol for Study of CB₁ Antagonistic Compound plus Lipase Inhibitor Effects on Obesity

The beneficial pharmacological effects of the combination of a CB₁ antagonist with a lipase inhibitor according to the invention can be shown by standard experimental animal models by measuring the influence of the administered combination of a CB₁ antagonist with a lipase inhibitor on the driving and characteristic parameters associated with obesity.

For investigation of the influence of the combination of a CB₁ antagonist with a lipase inhibitor on obesity the body weight gain in rats may be measured as a pharmacological indicator. Here fore, the following experimental protocol for rats may be applied:

The rats will have unlimited access to feed for two 2.5 h periods per day, during the dark phase of a reversed 12 h/12 h light cycle, e.g. lights are put on at 21.15 h and put off at 09.15 h. The rats will be offered a high fat, high sucrose diet (Western diet). The lipase inhibitor will be dosed immediately before the rats are fed. The CB₁ antagonist will be dosed 1 h before the lipase inhibitor is administered.

As an example, the following daily dosing schedule is applicable for a given period of e.g. days, weeks or months:

The CB₁ antagonist, in particular the CB₁ antagonisic compound of formula (I) as defined above, or a vehicle dose is administered (po) in the morning at ca. 07.45 to 08.00 h. The lipase inhibitor, e.g. in particular orlistat, or a vehicle dose is administered (po) ca. 08.45 to 09.00 h. After medication the rats are set to ad-libitum feed from 09.15 to 11.45 h, followed by feed removal from about 11.45 to 14.45 h. Another dose of lipase inhibitor, e.g. in particular orlistat, or vehicle (Labrasol) dose is administered (po) in the afternoon at about 14.15 to 14.30 h, followed by ad-libitum feed from 14.45 to 17.15 h. Thereafter, the rats are set to feed deprivation from 17.15-09.15 h.

The experimental protocol results will compare daily food intake and body weight gain as indicators for the effects of the combination treatment on obesity during the experimental phase. In addition to the before given parameters it may be desired to also collect faeces and to estimate fat digestibility. Eventually it may be also desired to perform a carcass analysis.

Furthermore, after finishing the experimental feeding and administering phase, biochemical parameters may be measured at slaughter of the rats.

For investigating the effects the total number of rats subject to the experimental protocol is divided into the following groups with approximately the same number of rats in each group:
1) Control group: The rats receive only vehicle according to the protocol to simulate administration (placebo group).
2) CB₁ group: The rats receive a CB₁ antagonist in a vehicle.
3) LI group: The rats receive as lipase inhibitor (“LI”) e.g. the compound orlistat in a vehicle.
4) CB₁+LI group (combination group): The rats receive a CB₁ antagonist in a vehicle and as lipase inhibitor (“LI”) e.g. the compound orlistat in a vehicle.

The results of this protocol and the respective investigations show the beneficial suitability of the combination of a CB₁ antagonist and a lipase inhibitor in the treatment and/or prophylaxis of obesity.

Preparation of Compounds of Formula (V)

The 1H-1,2,4-triazole-carboxamide derivatives of formula (V), which are potent cannabinoid-CB₁ receptor agonists, partial agonists, inverse agonists or antagonists, useful for the treatment of psychiatric and neurological disorders, as well as other diseases involving cannabinoid-CB₁ neurotransmission, are defined above in the specification.

1,5-Diaryl-1H-1,2,4-triazole-3-carboxamide derivatives have been described in EP 0346620 and GB 2120665 as herbicides. Recently 1,2,4-triazoles were described as potential agonists and antagonists of cannabinoid-CB₁ and -CB₂ receptors (Jagerovic, N. et al., Drugs Fut. 2002, 27(Suppl. A): XVIIth Int. Symp. on Medicinal Chemistry, P 284)

A group of four 1,5-diaryl-1H-1,2,4-triazole-3-carboxamide derivatives in which the amide N-atom is part of an unsubstituted piperidinyl or morpholinyl group is described by D. Clerin and J. P. Fleury in Bull. Soc. Chim. Fr., 1974,1-2, Pt. 2, 211-217.

1-(4-Methylphenyl)-5-phenyl-N-(2-pyridyl)-1H-1,2,4-triazole-3-carboxamide is described by M. H. Elnagdi et al. in Heteroatom Chem., 1995, 6, 589-592.

A group of four 1,5-diaryl-N-(2-pyridyl)-1H-1,2,4-triazole-3-carboxamides is described by A. H. Harhash et al. in Indian J. Chem., 1976, 14B, 268-272.

Suitable synthetic routes for the compounds of formula (V) used in the invention are the following:

Synthetic Route A

Step 1: Ester hydrolysis of a compound having formula (S-II) wherein R₆ represents a branched or unbranched (C₁₋₄)-alkyl group or a benzyl group, 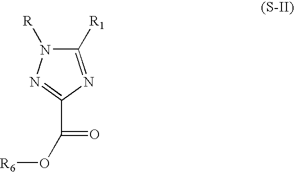
yields a compound having formula (S-III) 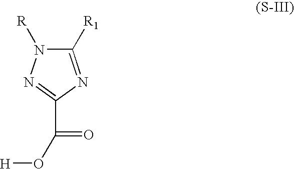
wherein R and R₁ have the meanings as described above.

The compounds of the invention having formula (S-II), wherein R₆ represents a branched or unbranched alkyl group (C₁₋₄) or benzyl group can be obtained according to methods known, for example:
a) Sawdey, G. W. J. Am. Chem. Soc. 1957, 79, 1955
b) Czollner, L. et al., Arch. Pharm. (Weinheim) 1990, 323, 225
c) Eicher, T. and Hauptmann, S. The Chemistry of Heterocycles, Thieme Verlag, Stuttgart, 1995 (ISBN 313 100511 4), p. 208-212.

Step 2: Reaction of a compound having formula (S-III) with a compound having formula R₂R₃NH wherein R₂ and R₃ have the meanings as described above via activating and coupling methods such as formation of an active ester, or in the presence of a coupling reagent such as DCC, HBTU, BOP, CIP (2-chloro-1,3-dimethylimidazolinium hexafluorophosphate) or PyAOP (7-azabenzotriazol-1-yloxytris(pyrrolidino)phosphonium hexafluorophosphate). Activating and coupling methods of this type are described in
a) M. Bodanszky and A. Bodanszky: The Practice of Peptide Synthesis, Springer-Verlag, New York, 1994; ISBN: 0-387-57505-7;
b) K. Akaji et al., Tetrahedron Lett. (1994), 35, 3315-3318);
c) F. Albericio et al., Tetrahedron Lett. (1997), 38, 4853-4856).
   This reaction gives a 1H-1,2,4-triazole derivative having formula (V).
   Synthetic Route B

A compound having formula (S-III) is reacted with a halogenating agent such as thionyl chloride (SOCl₂) or oxalyl chloride. This reaction yields the corresponding carbonyl chloride (acid chloride) (S-IV).

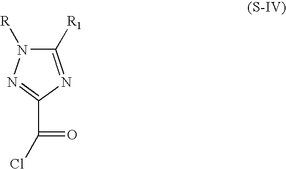

Reaction of a compound having formula (S-IV) with a compound having formula R₂R₃NH wherein R₂ and R₃ have the meanings as described above gives a 1H-1,2,4-triazole derivative having formula (V).

Synthetic Route C

A compound having formula (S-II) is reacted in an amidation reaction with a compound having formula R₂R₃NH wherein R₂ and R₃ have the meanings as described hereinabove to give a 1H-1,2,4-triazole derivative having formula (V). Such amidation reactions can be promoted by the use of trimethylaluminum AI(CH₃)₃ (For more information on aluminum-mediated conversion of esters to amides, see: J. I. Levin, E. Turos, S. M. Weinreb, Synth Commun. (1982), 12, 989-993.)

### EXAMPLE I

Part A: To a stirred solution of dimethyl aminomalonate hydrochloride (25 gram, 0.136 mol) in dichloromethane (200 mL) triethylamine (41.4 mL, 2.2 molar equivalent) is added at 0° C. 4-Chlorobenzoyl chloride (23.8 gram, 0.136 mol) is slowly added and the resulting solution is allowed to stand at room temperature overnight. Water is added and the organic layer is separated. The water layer is extracted twice with dichloromethane. The collected organic layers are washed with water, dried over MgSO₄, filtered and concentrated in vacuo. The residue is recrystallised from methanol (400 mL) to give dimethyl 2-(4-chlorobenzoylamino)malonate (30.5 gram, 79% yield). Melting point: 146-148° C. ¹H-NMR (200 MHz, CDCl₃): [symbol] 3.86 (s, 6H), 5.38 (d, J=6 Hz, 1H), 7.15 (br d, J=6 Hz, 1H), 7.43 (d, J=8 Hz, 2H), 7.79 (d, J=8 Hz, 2H).

Part B: To a stirred suspension of 2,4-dichloroaniline (19.44 gram, 0.12 mol) in concentrated HCl (25 mL) and acetic acid (75 mL) at 0° C. is added a solution of NaNO₂ (9.0 gram, 0.13 mol) in water (50 mL) and the resulting solution is stirred for 15 minutes. A solution of dimethyl 2-(4-chlorobenzoylamino)-malonate (28.55 gram, 0.10 mol) in acetone (200 mL) is slowly added while keeping the temperature below 0° C. A solution of K₂CO₃ (120 gram) in water (200 mL) is slowly added and the resulting black mixture is stirred for 30 minutes at 0° C. The mixture is extracted three times with EtOAc. The collected organics are washed with water, aqueous NaHCO₃ and water, respectively, dried over MgSO₄, filtered and concentrated in vacuo. The residue is dissolved in methanol (500 mL) and a solution of sodium (1 gram) in methanol (75 mL) is added. The resulting stirred mixture is allowed to stand overnight at room temperature and cooled in a refrigerator. The formed precipitate is collected by filtration and washed with methanol to give methyl 5-(4-chlorophenyl)-1-(2,4-dichlorophenyl)-1H-1,2,4-triazole-3-carboxylate (11.4 gram, 30% yield). Melting point: 153-154° C. ¹H-NMR (200 MHz, CDCl₃): [symbol] 4.07 (s, 3H), 7.28-7.60 (m, 7H).

Part C: To a stirred suspension of methyl 5-(4-chlorophenyl)-1-(2,4-dichlorophenyl)-1H-1,2,4-triazole-3-carboxylate (11.3 gram, 0.0295 mol) in methanol (100 mL) is added KOH (45% aqueous solution, 7.5 mL) and the resulting mixture is heated at reflux temperature for 4 hours. The mixture is concentrated in vacuo and water (150 mL) and concentrated HCl are added. The yellow precipitate is collected by filtration, washed with water and dried in vacuo to give 5-(4-chlorophenyl)-1-(2,4-dichlorophenyl)-1H-1,2,4-triazole-3-carboxylic acid (10.0 gram, 92% yield). Melting point: 141-144° C. (decomposition).

Part D: To a stirred solution of 5-(4-chlorophenyl)-1-(2,4-dichlorophenyl)-1H-1,2,4-triazole-3-carboxylic acid (1.48 gram, 4.0 mmol) in acetonitrile (20 mL) is successively added diisopropylethylamine (DIPEA) (1.5 mL, 2.1 molar equivalent), O-benzotriazol-1-yl-N,N,N′,N′-tetramethyluronium hexafluorophos-phate (HBTU) (1.66 gram, 1.1 molar equivalent) and 1-aminopiperidine (0.44 gram, 1.1 molar equivalent). After stirring overnight an aqueous NaHCO₃ solution is added. The resulting mixture is three times extracted with dichloromethane. The combined organic layers are washed with water, dried over Na₂SO₄, filtered and concentrated in vacuo to give a crude oil (3.6 gram). This oil is further purified by flash chromatography (silica gel; EtOAc/petroleum ether (40-60° C.)=7/3 (v/v)). The purified material is treated with ethanolic HCl (1 M solution) to give 5-(4-chlorophenyl)-1-(2,4-dichlorophenyl)-N-(piperidin-1-yl)-1H-1,2,4-triazole-3-carboxamide hydrochloride (1.50 gram, 77% yield). Melting point: 238-240° C. (decomposition). ¹H-NMR (400 MHz, DMSO-d₆): [symbol] 1.46-1.54 (m, 2H), 1.78-1.85 (m, 4H), 3.22-3.28 (m, 4H), 7.50 (s, 4H), 7.70 (dd, J=8 and 2 Hz, 1H), 7.85-7.87 (m, 1H), 7.91 (d, J=8 Hz, 1H), (NH not visible).

Analogously were prepared the examples 2-18:
2. 5-(4-Chlorophenyl)-1-(2,4-dichlorophenyl)-N-(pyrrolidin-1-yl)-1H-1,2,4-triazole-3-carboxamide hydrochloride. Melting point: 248-255° C. (decomposition).
3. 5-(4-Chlorophenyl)-N-cyclohexyl-1-(2,4-dichlorophenyl)-1H-1,2,4-triazole-3-carboxamide. Melting point: 186-188° C.
4. N-t-Butoxy-5-(4-chlorophenyl)-1-(2,4-dichlorophenyl)-1H-1,2,4-triazole-3-carboxamide. Melting point: 150-152° C.
5. 5-(4-Chlorophenyl)-1-(2,4-dichlorophenyl)-N-(n-pentyl)-1H-1,2,4-triazole-3-carboxamide. ¹H-NMR (400 MHz, CDCl₃): [symbol] 0.92 (t, J=7 Hz, 3H), 1.35-1.44 (m, 4H), 1.62-1.70 (m, 2H), 3.48-3.56 (m, 2H), 7.20-7.25 (m, 1H), 7.34 (dt, J=8 and 2 Hz, 2H), 7.42-7.50 (m, 4H), 7.54 (d, J=2 Hz, 1H).
6. 5-(4-Chlorophenyl)-1-(2,4-dichlorophenyl)-N-(morpholin-4-yl)-1H-1,2,4-triazole-3-carboxamide. Melting point: 184-186° C.
7. 1-(4-Chlorophenyl)-5-(2,4-dichlorophenyl)-N-(piperidin-1-yl)-1H-1,2,4-triazole-3-carboxamide hydrochloride. Melting point: 234-237° C. (decomposition).
8. 1-(4-Chlorophenyl)-5-(2,4-dichlorophenyl)-N-(pyrrolidin-1-yl)-1H-1,2,4-triazole-3-carboxamide hydrochloride. Melting point: 234-236° C. (decomposition).
9. 1-(4-Chlorophenyl)-N-cyclohexyl-5-(2,4-dichlorophenyl)-1H-1,2,4-triazole-3-carboxamide. ¹H-NMR (400 MHz, CDCl₃): □ 1.14-1.81 (m, 8H), 2.02-2.10 (m, 2H), 4.00-4.11 (m, 1H), 7.08 (br d, J˜7 Hz, 1H), 7.26 (br d, J˜8 Hz, 2H), 7.34 (br d, J˜8 Hz, 2H), 7.40 (dd, J=8 and 2 Hz, 1H), 7.44-7.48 (m, 2H).
10. N-t-Butoxy-1-(4-chlorophenyl)-5-(2,4-dichlorophenyl)-1H-1,2,4-triazole-3-carboxamide. ¹H-NMR (400 MHz, CDCl₃): □ 1.38 (s, 9H), 7.25 (br d, J˜8 Hz, 2H), 7.35 (br d, J˜8 Hz, 2H), 7.41 (dd, J˜8 and 2 Hz, 1H), 7.44-7.48 (m, 2H), 9.18, br s, 1H).
11. 1-(4-Chlorophenyl)-5-(2,4-dichlorophenyl)-N-(n-pentyl)-1H-1,2,4-triazole-3-carboxamide. ¹H-NMR (400 MHz, CDCl₃): □ 0.91 (t, J=7 Hz, 3H), 1.35-1.41 (m, 4H), 1.60-1.70 (m, 2H), 3.48-3.56 (m, 2H), 7.21 (br t, J˜7 Hz, 1H), 7.26 (br d, J˜8 Hz, 2H), 7.34 (br d, J˜8 Hz, 2H), 7.40 (dd, J=8 and 2 Hz, 1H), 7.44-7.48 (m, 2H).
12. 1-(4-Chlorophenyl)-5-(2,4-dichlorophenyl)-N-(morpholin-4-yl)-1H-1,2,4-triazole-3-carboxamide hydrochloride. Melting point: 224-226° C.
13. 1-(2,4-Dichlorophenyl)-5-(pyridin-2-yl)-N-(piperidin-1-yl)-1H-1,2,4-triazole-3-carboxamide. Melting point: 191-193° C.
14. 5-(2,4-Dichlorophenyl)-N-(piperidin-1-yl)-1-(4-(trifluoromethyl)phenyl)-1H-1,2,4-triazole-3-carboxamide. Melting point: 159-161° C.
15. 1′-[5-(2,4-dichlorophenyl)-1-(4-(trifluoromethyl)phenyl)-1H-1,2,4-triazol-3-yl)carbonyl]piperidine. Melting point: 155-156° C.
16. 1-(2,4-Dichlorophenyl)-N-(piperidin-1-yl)-5-(pyridin-3-yl)-1H-1,2,4-triazole-3-carboxamide. Melting point: 219° C.
17. 1-(4-Chlorophenyl)-5-(2,4-dichlorophenyl)-N-(5,5,5-trifluoropentyl)-1H-1,2,4-triazole-3-carboxamide. ¹H-NMR (400 MHz, CDCl₃): [symbol] 1.63-1.80 (m, 4H), 2.06-2.22 (m, 2H), 3.54 (q, J˜7 Hz, 2H), 7.26 (m, 3H), 7.34 (br d, J˜8 Hz, 2H), 7.40 (dd, J=8 and 2 Hz, 1H), 7.44-7.48 (m, 2H).
18. 1-(4-Chlorophenyl)-5-(2,4-dichlorophenyl)-N-(5-fluoropentyl)-1H-1,2,4-triazole-3-carboxamide. ¹H-NMR (400 MHz, CDCl₃): [symbol] 1.63-1.80 (m, 4H), 2.06-2.22 (m, 2H), 3.54 (q, J˜7 Hz, 2H), 7.22-7.28 (m, 3H), 7.34 (br d, J˜8 Hz, 2H), 7.40 (dd, J=8 and 2 Hz, 1H), 7.44-7.48 (m, 2H).

### EXAMPLE 19

Part A: 1-(Chlorophenyl)-5-(2,4-dichlorophenyl)-1H-1,2,4-triazole-3-carboxylic acid was prepared analogously to the procedure as described in Example 1, Part A-C by using dimethyl aminomalonate hydrochloride, 2,4-dichlorobenzoyl chloride and 4-chloroaniline as starting materials, respectively. Melting point: 102-104° C. ¹H-NMR (400 MHz, DMSO-d₆): [symbol] 7.36 (br d, J˜8 Hz, 2H), 7.50 (br d, J˜8 Hz, 2H), 7.59 (dd, J=8 and 2 Hz, 1H), 7.70 (d, J=2 Hz, 1H), 7.75 (d, J=8 Hz, 1H), OH proton is part of water peak at □ 3.4.

Analogously was 1-(chlorophenyl)-5-(2,5-dichlorophenyl)-1H-1,2,4-triazole-3-carboxylic acid prepared by using dimethyl aminomalonate hydrochloride, 2,5-dichlorobenzoyl chloride and 4-chloroaniline as starting materials, respectively. Melting point: 183-188° C. ¹H-NMR (400 MHz, DMSO-d₆): [symbol] 7.41 (br d, J˜8 Hz, 2H), 7.52 (br d, J˜8 Hz, 2H), 7.56 (d, J=8 Hz, 1H), 7.65 (dd, J=8 and 2 Hz, 1H), 7.88 (d, J=2 Hz, 1H), OH proton is part of water peak at □ 3.5.

Part B: To a stirred solution of 1-(chlorophenyl)-5-(2,4-dichlorophenyl)-1H-1,2,4-triazole-3-carboxylic acid (0.37 g, 1.00 mmol) in dichloromethane (10 mL) is added oxalyl chloride (0.254 g, 2.00 mmol). The resulting mixture is concentrated in vacuo to give crude 1-(chlorophenyl)-5-(2,4-dichlorophenyl)-1H-1,2,4-triazole-3-carbonyl chloride.

Part C: The crude 1-(chlorophenyl)-5-(2,4-dichlorophenyl)-1H-1,2,4-triazole-3-carbonyl chloride is dissolved in tetrahydrofuran (THF) (10 mL). 2,3-Dihydro-1H-inden-2-ylamine (0.40 g, 3.00 mmol) is added and the resulting solution is stirred for 42 hours at 25° C. The mixture is concentrated in vacuo and the residue is purified by preparative liquid chromatography to give pure 1-(4-chlorophenyl)-5-(2,4-dichlorophenyl)-N-(2,3-dihydro-1H-inden-2-yl)-1H-1,2,4-triazole-3-carboxamide (393 mg, 81% yield). MS (ESI+) 485.6. ¹H-NMR (400 MHz, DMSO-d₆): 3.06 (dd, J=16 and 8 Hz, 2H), 3.21 (dd, J=16 and 8 Hz, 2H), 4.71-4.82 (m, 1H), 7.12-7.16 (m, 2H), 7.19-7.24 (m. 2H), 7.39 (br d, J˜8 Hz, 2H), 7.52 (br d, J˜8 Hz, 2H), 7.60 (dd, J=8 and 2 Hz, 1H), 7.71 (d, J=2 Hz, 1H), 7.79 (d, J=8 Hz, 1H), 8.93-8.97 (m, 1H, NH).

Analogously were prepared the examples 20-43:
20. 1-(4-Chlorophenyl)-5-(2,4-dichlorophenyl)-N-(1-ethynylcyclohexyl)-1H-1,2,4-triazole-3-carboxamide. MS (ESI⁺) 473.3.
21. 1-(4-Chlorophenyl)-5-(2,4-dichlorophenyl)-N-(2-methylcyclohexyl)-1H-1,2,4-triazole-3-carboxamide. MS (ESI⁺) 465.5.
22. 1-(4-Chlorophenyl)-5-(2,4-dichlorophenyl)-N-(4-methylcyclohexyl)-1H-1,2,4-triazole-3-carboxamide. MS (ESI⁺) 465.5.
23. 1-(4-Chlorophenyl)-5-(2,4-dichlorophenyl)-N-cyclooctyl-1H-1,2,4-triazole-3-carboxamide. MS (ESI⁺) 477.3.
24. 1-(4-Chlorophenyl)-5-(2,4-dichlorophenyl)-N-(azepan-1-yl)-1H-1,2,4-triazole-3-carboxamide. MS (ESI⁺) 466.4.
25. 1-(4-Chlorophenyl)-5-(2,4-dichlorophenyl)-N-cycloheptyl-1H-1,2,4-triazole-3-carboxamide. MS (ESI⁺) 465.5.
26. N-t-Butyl-1-(4-chlorophenyl)-5-(2,4-dichlorophenyl)-1H-1,2,4-triazole-3-carboxamide. MS (ESI⁺) 425.4.
27. 1-(4-Chlorophenyl)-5-(2,4-dichlorophenyl)-N-(1,1-diethylprop-2-yn-1-yl)-1H-1,2,4-triazole-3-carboxamide. MS (ESI⁺) 461.5.
28. 1-(4-Chlorophenyl)-5-(2,4-dichlorophenyl)-N-(2,2,2-trifluoroethyl)-1H-1,2,4-triazole-3-carboxamide. MS (ESI⁺) 451.3.
29. 1-(4-Chlorophenyl)-5-(2,4-dichlorophenyl)-N-(exo-bicyclo[2.2.1]hept-2-yl)-1H-1,2,4-triazole-3-carboxamide. MS (ESI⁺) 461.5.
30. 1-(4-Chlorophenyl)-5-(2,4-dichlorophenyl)-N-(4-(2-propyl)piperazin-1-yl)-1H-1,2,4-triazole-3-carboxamide. MS (ESI⁺) 480.3. ¹H-NMR (400 MHz, DMSO-d₆): 1.00 (d, J=7 Hz, 6H), 2.46-2.56 (m, 4H), 2.72 (septet, J=7 Hz, 1H), 3.66-3.74 (m, 4H), 7.36 (br d, J=8 Hz, 2H), 7.51 (br d, J=8 Hz, 2H), 7.59 (dd, J=8 and 2 Hz, 1H), 7.72 (d, J=2 Hz, 1H), 7.75 (d, J=8 Hz, 1H).
31. 1-(4-Chlorophenyl)-5-(2,4-dichlorophenyl)-N-(hexahydrocyclopenta[c]pyrrol-2(1H)-yl)-1H-1,2,4-triazole-3-carboxamide. MS (ESI⁺) 476.4.
32. 1-(4-Chlorophenyl)-5-(2,4-dichlorophenyl)-N-pentyl-1H-1,2,4-triazole-3-carboxamide. MS (ESI⁺) 435.5.
33. 1-(4-Chlorophenyl)-5-(2,4-dichlorophenyl)-N-(2,2-dimethylpropyl)-1H-1,2,4-triazole-3-carboxamide. MS (ESI⁺) 439.6.
34. 1-(4-Chlorophenyl)-5-(2,4-dichlorophenyl)-N-(3-(trifluoromethyl)phenyl)-1H-1,2,4-triazole-3-carboxamide. MS (ESI⁺) 511.7.
35. 1′-[1-(4-Chlorophenyl)-5-(2,4-dichlorophenyl)-1H-1,2,4-triazol-3-yl)carbonyl]-1,4′-bipiperidine. MS (ESI⁺) 520.5.
36. 1-(4-Chlorophenyl)-N-(4-chlorophenyl)-5-(2,5-dichlorophenyl)-N-methyl-1H-1,2,4-triazole-3-carboxamide. MS (ESI⁺) 491.4.
37. 1-(4-Chlorophenyl)-5-(2,5-dichlorophenyl)-N-(1-ethynylcyclohexyl)-1H-1,2,4-triazole-3-carboxamide. MS (ESI⁺) 473.4.
38. 1-(4-Chlorophenyl)-5-(2,5-dichlorophenyl)-N-(2-methylcyclohexyl)-1H-1,2,4-triazole-3-carboxamide. MS (ESI⁺) 465.5.
39. 1-(4-Chlorophenyl)-5-(2,5-dichlorophenyl)-N-(4-methylcyclohexyl)-1H-1,2,4-triazole-3-carboxamide. MS (ESI⁺) 465.6.
40. 1-(4-Chlorophenyl)-5-(2,5-dichlorophenyl)-N-cyclooctyl-1H-1,2,4-triazole-3-carboxamide. MS (ESI⁺) 477.3.
41. 1-(4-Chlorophenyl)-5-(2,5-dichlorophenyl)-N-cycloheptyl-1H-1,2,4-triazole-3-carboxamide. MS (ESI⁺) 465.6.
42. 1-(4-Chlorophenyl)-5-(2,5-dichlorophenyl)-N-cyclopentyl-1H-1,2,4-triazole-3-carboxamide. MS (ESI⁺) 435.5.
43. 1-(4-Chlorophenyl)-5-(2,5-dichlorophenyl)-N-(2,2-dimethylpropyl)-1H-1,2,4-triazole-3-carboxamide. MS (ESI⁺) 439.6.

Pharmacological test results of a subset of the compounds of the invention, obtained with the assays described above, are given in the table below:

Human cannabinoid-CB₁ receptors

| | In vitro affinity | In vitro antagonism |
|---|---|---|
| Example | pK₁ value | pA₂ value |
| Example 2 | 6.6 | 7.2 |
| Example 3 | 6.9 | 8.7 |
| Example 5 | 6.9 | |
| Example 9 | 7.4 | 8.2 |
| Example 11 | 6.3 | |

1. Use of a compound with CB₁-receptor activity of formula (I), (II), (III), (IV) and/or (V), a prodrug thereof, a tautomer thereof or a salt thereof, in the manufacture of medicaments for the treatment and/or prophylaxis of CB₁ receptor related diseases in juvenile patients and/or for the treatment and/or prophylaxis of drug induced obesity in juvenile, as well as in adolescent, patients.

2. Use of a compound with CB₁-receptor activity according to claim 1, wherein the compound with CB₁-receptor activity is selected from the group of 4,5-dihydro-1H-pyrazole derivatives of the formula (I) and/or (III), 1H-Imidazole derivatives of the formula (II), thiazole derivatives of the formula (IV) and/or 1H-1,2,4-triazole-3-carboxamide derivatives of the formula (V), characterized in that:
a) the compounds of formula (I) are 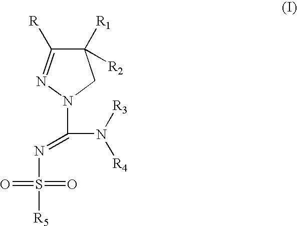
wherein
R and R₁ independently represent phenyl, thienyl or pyridyl which groups may be substituted with 1, 2, 3 or 4 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
R₃ represents a hydrogen atom or a branched or unbranched C₁₋₈ alkyl group or a C₃₋₇ cycloalkyl group which alkyl group or cycloalkyl group may be substituted with a hydroxy group,
R₄ represents a C₂₋₁₀ branched or unbranched heteroalkyl group, C₃₋₈ non-aromatic heterocycloalkyl group or C₄₋₁₀ non-aromatic heterocycloalkyl-alkyl group which groups contain one or more heteroatoms from the group (O, N, S) or a —SO₂— group, which C₂₋₁₀ branched or unbranched heteroalkyl group, C₃₋₈ non-aromatic heterocycloalkyl group or C₄₋₁₀ non-aromatic heterocycloalkyl-alkyl group may be substituted with a keto group, trifluoromethyl group, C₁₋₃ alkyl group, hydroxy, amino, monoalkylamino, or dialkylamino group or a fluoro atom, or R₄ represents an amino, hydroxy, phenoxy or benzyloxy group, or R₄ represents a C₁₋₈ alkoxy, C₃₋₈ alkenyl, C₅₋₈ cycloalkenyl or C₆₋₉ cycloalkenylalkyl group which groups may contain a sulphur, nitrogen or oxygen atom, a keto group or —SO₂— group, which alkoxy, alkenyl and cycloalkenyl groups may be substituted with a hydroxy group, a trifluoromethyl group, an amino group, a monoalkylamino group or dialkylamino group or a fluoro atom, or R₄ represents a C₂₋₅ alkyl group which alkyl group contains a fluoro atom, or R₄ represents an imidazolylalkyl group, benzyl, pyridylmethyl, phenethyl or thienyl group, or R₄ represents a substituted phenyl, benzyl, pyridyl, thienyl, pyridylmethyl or phenethyl group wherein the aromatic rings are substituted with 1, 2 or 3 of the substituents Y, wherein Y has the meaning as indicated above, or when R₃ is H or methyl, R₄ may represent a group NR₆R₇
wherein
R₆ and R₇ are the same or different and represent C₂₋₄ alkyl, C₂₋₄ trifluoroalkyl or R₆ represents a methyl group with the proviso that R₇ represents a C₂₋₄ alkyl group, or R₆ and R₇—together with the nitrogen atom to which they are bonded—form a saturated or unsaturated heterocyclic moiety having 4 to 8 ring atoms which heterocyclic moiety may contain an oxygen or sulphur atom or a keto group or —SO₂— group or an additional nitrogen atom, which saturated or unsaturated heterocyclic moiety may be substituted with a C₁₋₄ alkyl group, or
R₃ and R₄ together with the nitrogen atom to which they are bonded form a saturated or unsaturated, monocyclic or bicyclic heterocyclic moiety having 4 to 10 ring atoms, which heterocyclic moiety may contain one or more atoms from the group (O, N, S) or a keto group or —SO₂— group, which moiety may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group,
R₅ represents benzyl, phenyl, thienyl or pyridyl which may be substituted with 1, 2, 3 or 4 substituents Y, wherein Y has the meaning as indicated above, which can be the same or different, or R₅ represents C₁₋₈ branched or unbranched alkyl, C₃₋₈ alkenyl, C₃₋₁₀ cycloalkyl, C₅₋₁₀ bicycloalkyl, C₆₋₁₀ tricycloalkyl or C₅₋₈ cycloalkenyl or R₅ represents naphtyl.
b) the compounds of formula (II) are 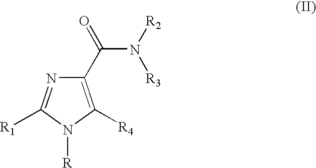
wherein
R represents phenyl, thienyl, 2-pyridinyl, 3-pyridinyl, 4-pyridinyl, pyrimidinyl, pyrazinyl, pyridazinyl or triazinyl, which groups may be substituted with 1, 2, 3 or 4 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkoxycarbonyl, carboxyl, cyano, carbamoyl and acetyl, or R represents naphtyl, with the proviso that when R is 4-pyridinyl, R₄ represents a halogen atom or a cyano, carbamoyl, formyl, acetyl, trifluoroacetyl, fluoroacetyl, propionyl, sulfamoyl, methanesulfonyl, methylsulfanyl or branched or unbranched C₁₋₄ alkyl group, which C₁₋₄ alkyl group may be substituted with 1-3 fluoro atoms or with a bromo, chloro, iodo, cyano or hydroxy group,
R₁ represents phenyl or pyridinyl, which groups may be substituted with 1-4 substituents Y, which can be the same or different, wherein Y has the above mentioned meaning, or R₁ represents pyrimidinyl, pyrazinyl, pyridazinyl or triazinyl, which groups may be substituted with 1-2 substituents Y, which can be the same or different or R₁ represents a five-membered aromatic heterocyclic ring having one or two heteroatoms from the group (N, O, S), which heteroatoms can be the same or different, which five-membered aromatic heterocyclic ring may be substituted with 1-2 substituents Y, which can be the same or different or R₁ represents naphtyl,
R₂ represents H, branched or unbranched C₁₋₈ alkyl, C₃₋₈ cycloalkyl, C₃₋₈ alkenyl, C₅₋₈ cycloalkenyl which groups may contain a sulfur, oxygen or nitrogen atom,
R₃ represents branched or unbranched C₂₋₈ alkyl, C₁₋₈ alkoxy, C₅₋₈ cycloalkyloxy, C₃₋₈ cycloalkyl, C₅₋₁₀ bicycloalkyl, C₆₋₁₀ tricycloalkyl, C₃₋₈ alkenyl, C₅₋₈ cycloalkenyl, which groups may optionally contain one or more heteroatoms from the group (O, N, S) and which groups may be substituted with a hydroxy group or 1-2 C₁₋₃ alkyl groups or 1-3 fluoro atoms, or R₃ represents a benzyl or phenethyl group which aromatic rings may be substituted with 1-5 substituents Z, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkylsulfonyl, dimethyl-sulfamido, C₁₋₃-alkoxycarbonyl, carboxyl, trifluoromethylsulfonyl, cyano, carbamoyl, sulfamoyl and acetyl, or R₃ represents a phenyl or pyridinyl group, which groups are substituted with 1-4 substituents Z, wherein Z has the meaning as indicated above, or R₃ represents a pyridinyl group, or R₃ represents a phenyl group, with the proviso that R₄ represents a halogen atom or a cyano, carbamoyl, formyl, acetyl, trifluoroacetyl, fluoroacetyl, propionyl, sulfamoyl, methanesulfonyl, methylsulfanyl or C₁₋₄ alkyl group, which C₁₋₄ alkyl group may be substituted with 1-3 fluoro atoms or with a bromo, chloro, iodo, cyano or hydroxy group, or R₃ represents a group NR₅R₆ with the proviso that R₂ represents a hydrogen atom or a methyl group, wherein
R₅ and R₆ are the same or different and represent branched or unbranched C₁₋₄ alkyl, or R₅ and R₆—together with the nitrogen atom to which they are bonded—form a saturated or unsaturated, monocyclic or bicyclic heterocyclic group having 4 to 10 ring atoms which heterocyclic group contains one or two heteroatoms from the group (N, O, S), which heteroatoms can be the same or different, which heterocyclic group may be substituted with a C₁₋₃ alkyl group or a hydroxy group, or R₂ and R₃—together with the nitrogen atom to which they are bonded—form a saturated or unsaturated heterocyclic group having 4 to 10 ring atoms which heterocyclic group contains one or two heteroatoms from the group (N, O, S), which heteroatoms can be the same or different, which heterocyclic group may be substituted with a C₁₋₃ alkyl group or a hydroxy group,
R₄ represents a hydrogen or halogen atom or a cyano, carbamoyl, formyl, acetyl, trifluoroacetyl, fluoroacetyl, propionyl, sulfamoyl, methanesulfonyl, methylsulfanyl or branched or unbranched C₁₋₄ alkyl group, which C₁₋₄ alkyl group may be substituted with 1-3 fluoro atoms or with a bromo, chloro, iodo, cyano or a hydroxy group,
c) the compounds of formula (III) are 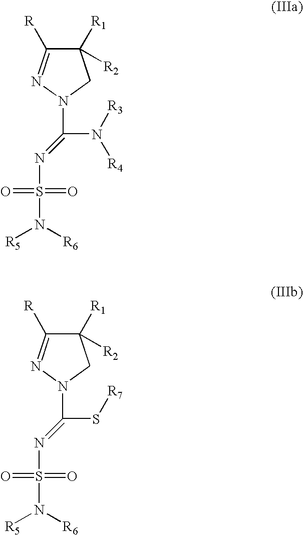
wherein
R and R₁ independently represent phenyl, thienyl or pyridyl which groups may be substituted with 1, 2 or 3 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
R₃ represents a hydrogen atom or a branched or unbranched C₁₋₈ alkyl group or a C₃₋₇ cycloalkyl group which alkyl group or cycloalkyl group may be substituted with a hydroxy group,
R₄ represents a hydrogen atom or a branched or unbranched C₁₋₈ alkyl, C₃₋₈ cycloalkyl, C₂₋₁₀ heteroalkyl, C₃₋₈ nonaromatic heterocycloalkyl or C₄₋₁₀ nonaromatic heterocycloalkyl-alkyl moiety which moieties may contain one or more heteroatoms from the group (O, N, S), which moieties may be substituted with a keto group, trifluoromethyl group, C₁₋₃ alkyl group, hydroxy, amino, monoalkylamino, or dialkylamino group or a fluoro atom, or R₄ represents an amino, hydroxy, phenoxy or benzyloxy group or R₄ represents a branched or unbranched C₁₋₈ alkoxy, C₃₋₈ alkenyl, C₅₋₈ cycloalkenyl or C₆₋₉ cycloalkenylalkyl group which groups may contain a sulphur, nitrogen or oxygen atom, a keto group or —SO₂— group which C₁₋₈ alkoxy, C₃₋₈ alkenyl, C₅₋₈ cycloalkenyl or C₆₋₉ cycloalkenylalkyl groups may be substituted with a hydroxy group, a trifluoromethyl group, an amino group, a monoalkylamino group or dialkylamino group or a fluoro atom, or R₄ represents a phenyl, benzyl, pyridyl, thienyl, pyridylmethyl or phenethyl group wherein the aromatic rings may be substituted with 1, 2 or 3 of the substituents Y, wherein Y has the meaning as indicated above, or R₄ represents a group NR₈R₉ with the proviso that R₃ represents a hydrogen atom or a methyl group and wherein R₈ and R₉ are the same or different and represent C₁₋₄ alkyl or C₂₋₄ trifluoroalkyl or R₈ and R₉—together with the nitrogen atom to which they are bonded—form a saturated or un-saturated heterocyclic moiety having 4 to 8 ring atoms which heterocyclic moiety may contain an oxygen or sulphur atom or a keto group or —SO₂— group or an additional nitrogen atom, which saturated or unsaturated heterocyclic moiety may be substituted with a C₁₋₄ alkyl group or R₃ and R₄—together with the nitrogen atom to which they are bonded—form a saturated or unsaturated, monocyclic or bicyclic heterocyclic moiety having 4 to 10 ring atoms, which heterocyclic moiety may contain one or more atoms from the group (O, N, S) or a keto group or —SO₂— group, which moiety may be substituted with a C₁₋₄ alkyl, hydroxyalkyl, phenyl, thienyl, pyridyl, amino, monoalkylaminoalkyl, dialkylaminoalkyl, monoalkylamino, dialkylamino, aminoalkyl, azetidinyl, pyrrolidinyl, piperidinyl or hexahydro-1H-azepinyl group,
R₅ and R₆ independently of each other represent a hydrogen atom or a branched or unbranched C₁₋₈ alkyl or alkenyl group which groups may contain one or more heteroatoms from the group (O, N, S), a keto group or a —SO₂— group and which groups may be substituted with a hydroxy or amino group, or R₅ and R₆ independently of each other represent a C₃₋₈ cycloalkyl group or C₃₋₈ cycloalkenyl group which may contain one or more ring heteroatoms from the group (O, N, S) or the —SO₂— group and which groups may be substituted with a hydroxy group, alkyl (C₁₋₃), the —SO₂— group, the keto group, amino group, monoalkylamino group (C₁₋₃) or dialkylamino group (C₁₋₃), or R₅ represents a naphtyl group or a phenyl group which phenyl group may be substituted with 1, 2 or 3 substituents Y wherein Y has the meaning as described hereinabove, with the proviso that R₆ represents a hydrogen atom, or a branched or unbranched alkyl group (C₁₋₅) which alkyl group may contain one or more heteroatoms from the group (O, N, S) or the —SO₂— group and which alkyl group may be substituted with a hydroxy, keto or amino group, or R₅ and R₆—together with the nitrogen atom to which they are bonded—form a monocyclic, bicyclic or tricyclic alkyl or alkenyl group which may contain ring heteroatoms from the group (O, N, S), the keto or the SO₂ group and which monocyclic, bicyclic or tricyclic alkyl or alkenyl group may be substituted with a hydroxy group, alkyl (C₁₋₃) group, SO₂ group, keto group, amino group, monoalkylamino group (C₁₋₃), dialkylamino group (C₁₋₃), pyrrolidinyl group or piperidinyl group, which monocyclic, bicyclic or tricyclic alkyl or alkenyl group may contain an annelated phenyl group which annelated phenyl group may be substituted with 1 or 2 substituents Y, wherein Y has the meaning as described herein above,
R₇ represents branched or unbranched C₁₋₃ alkyl,
d) the compounds of formula (IV) are 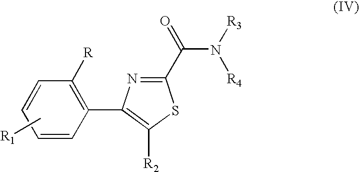
wherein
R represents a hydrogen atom or a substituent X from the group branched or unbranched C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, branched or unbranched (C₁₋₃)-alkoxycarbonyl, trifluoromethylsulfonyl, sulfamoyl, branched or unbranched alkyl(C₁₋₃)sulfonyl, carboxyl, cyano, carbamoyl, branched or unbranched dialkyl(C₁₋₃) aminosulfonyl, branched or unbranched monoalkyl(C₁₋₃)-aminosulfonyl and acetyl,
R₁ is a hydrogen atom or represents 1-4 substituents X, wherein X has the abovementioned meaning,
R₂ represents a phenyl, thienyl, pyridyl or pyrimidinyl group, which groups may be substituted with 1-4 substituents X, wherein X has the abovementioned meaning or R₂ represents naphtyl,
R₃ represents a hydrogen atom or a branched or unbranched C₁₋₁₀ alkyl or cycloalkyl-alkyl group or a phenyl, benzyl or phenethyl group which aromatic rings may be substituted with 1-5 substituents Z, which can be the same or different, from the group branched or unbranched C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, branched or unbranched (C₁₋₃)-alkylsulfonyl, dimethylsulfamido, branched or unbranched C₁₋₃-alkoxycarbonyl, carboxyl, trifluoromethylsulfonyl, cyano, carbamoyl, sulfamoyl and acetyl, or R₃ represents a pyridyl or thienyl group,
R₄ represents branched or unbranched C₁₋₁₀ alkyl or cycloalkyl-alkyl group, branched or unbranched C₁₋₁₀ alkoxy, C₃₋₈ cycloalkyl, C₅₋₁₀ bicycloalkyl, C₋₆₁₀ tricycloalkyl, branched or unbranched C₃₋₁₀ alkenyl, C₅₋₈ cycloalkenyl, which groups may contain one or more heteroatoms from the group (O, N, S) and which groups may be substituted with a hydroxy group, 1-3 methyl groups, an ethyl group or 1-3 fluoro atoms, or R₄ represents a phenyl, benzyl or phenethyl group which aromatic rings may be substituted with 1-5 substituents Z, wherein Z has the abovementioned meaning, or R₄ represents a pyridyl or thienyl group, or R₄ represents a group NR₅R₆ wherein R₅ and R₆ together with the nitrogen atom to which they are attached form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or unbranched C₁₋₃ alkyl, hydroxy or trifluoromethyl group or a fluoro atom, or
R₃ and R₄—together with the nitrogen atom to which they are attached—form a saturated or unsaturated, monocyclic or bicyclic, heterocyclic group having 4 to 10 ring atoms, which heterocyclic group contains one or more heteroatoms from the group (O, N, S) and which heterocyclic group may be substituted with a branched or unbranched C₁₋₃ alkyl, hydroxy or trifluoromethyl group or a fluoro atom,
e) the compounds of formula (V) are 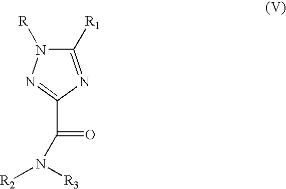
wherein
R and R₁ independently represent a phenyl, naphtyl, thienyl, pyridyl, pyrimidyl, pyrazinyl, pyridazinyl or triazinyl group, which groups may be substituted with 1-4 substituents X, which can be the same or different, from the group branched or unbranched (C₁₋₃)-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkoxycarbonyl, trifluoromethylsulfonyl, sulfamoyl, (C₁₋₃)-alkylsul-fonyl, carboxyl, cyano, carbamoyl, (C₁₋₃)-dialkylaminosulfonyl, (C₁₋₃)-monoalkylamino-sulfonyl and acetyl,
R₂ represents a hydrogen atom or a branched or unbranched C₁₋₈ alkyl or C₁₋₈ cycloalkyl-alkyl group or a phenyl, benzyl or phenethyl group which aromatic rings may be substituted with 1-4 substituents X, wherein X has the meaning as indicated above, or R₂ represents a pyridyl or thienyl group,
R₃ represents branched or unbranched C₁₋₈ alkyl, C₁₋₈ alkoxy, C₃₋₈ cycloalkyl, C₅₋₁₀ bicycloalkyl, C₆₋₁₀ tricycloalkyl, C₃₋₈ alkenyl, C₅₋₈ cycloalkenyl, which groups may optionally contain one or more heteroatoms from the group (O, N, S), which groups may be substituted with a hydroxy group, an ethynyl group or 1-3 fluoro atoms, or R₃ represents a phenyl, benzyl or phenethyl group which aromatic rings may be substituted with 1-4 substituents X, wherein X has the meaning as indicated above, or R₃ represents a pyridyl, pyrimidyl, pyrazinyl, pyridazinyl, triazinyl or thienyl group which heteroaromatic rings may be substituted with 1-2 substituents X, wherein X has the meaning as indicated above, or R₃ represents a group NR₄R₅ wherein R₄ and R₅, together with the nitrogen atom to which they are bonded, form a saturated or unsaturated, monocyclic or bicyclic, heterocyclic moiety having 4 to 10 ring atoms, which heterocyclic group contains one or two heteroatoms from the group N, O or S, which heteroatoms can be the same or different, which heterocyclic moiety may be substituted with a branched or unbranched C₁₋₃ alkyl, hydroxy or trifluoromethyl group or a fluoro atom, or
R₂ and R₃, together with the nitrogen atom to which they are bonded, form a saturated or unsaturated, monocyclic or bicyclic, heterocyclic moiety having 4 to 10 ring atoms, which heterocyclic group contains one or two heteroatoms from the group N, O or S, which heteroatoms can be the same or different, which heterocyclic moiety may be substituted with a branched or unbranched C₁₋₃ alkyl, hydroxy, piperidinyl or trifluoromethyl group or a fluoro atom.

3. Use of a compound with CB₁-receptor activity according to anyone of the claims 1 to 2, or a prodrug, tautomer or salt thereof, wherein the use is in the manufacture of a medicament for pediatric treatment and/or prophylaxis pertaining to psychiatric disorders such as psychosis, anxiety, depression, attention deficits, memory disorders, cognitive disorders, appetite disorders, obesity, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischemia, cerebral apoplexy, craniocerebral trauma, stroke, spinal cord injury, neuroinflammatory disorders, plaque sclerosis, viral encephalitis, demyelinisation related disorders, as well as for the pediatric treatment of pain disorders, including neuropathic pain disorders, and other diseases involving cannabinoid neurotransmission, including the pediatric treatment of septic shock, glaucoma, cancer, diabetes, emesis, nausea, asthma, respiratory diseases, gastrointestinal disorders, gastric ulcers, diarrhea and cardiovascular disorders.

4. Use of a compound with CB₁-receptor activity according to anyone of the claims 1 to 3, or a prodrug, tautomer or salt thereof, preferably of a CB₁ receptor antagonistic compound or a prodrug, tautomer or salt thereof, wherein the use is in the manufacture of a medicament for the treatment and/or prophylaxis of obesity in juvenile patients and/or drug induced obesity in juvenile, as well as adolescent, patients.

5. Use of a compound with CB₁-receptor activity as defined in claim 2, or a prodrug, tautomer or salt thereof, preferably of a CB₁ receptor antagonistic compound or a prodrug, tautomer or salt thereof, in combination with at least one lipase inhibiting compound in the manufacture of a medicament for the treatment and/or prophylaxis of obesity in adolescent or in juvenile patients and/or drug induced obesity in juvenile, as well as adolescent, patients.

6. Use of a compound with CB₁-receptor activity according to claims 5, wherein the compound with CB₁-receptor activity or a prodrug, tautomer or salt thereof, preferably the CB₁ receptor antagonistic compound or a prodrug, tautomer or salt thereof, is used in combination with at least one lipase inhibiting compound selected from the group of lipase inhibiting polymers, orlistat, panclicins, ATL-962 and lipstatin.

7. A pharmaceutical composition containing at least one compound with CB₁-receptor activity of formula (I), (II), (III), (IV) and/or (V), or a prodrug, tautomer or salt thereof, as an active component suited for the treatment and/or prophylaxis of CB₁ receptor related diseases in juvenile patients and/or for the treatment and/or prophylaxis of drug induced obesity in juvenile, as well as in adolescent, patients.

8. A pharmaceutical composition according to claim 7, wherein the compound with CB₁-receptor activity is selected from the group of 4,5-dihydro-1H-pyrazole derivatives of the formula (I) and/or (III), 1H-Imidazole derivatives of the formula (II), thiazole derivatives of the formula (IV) and/or 1H-1,2,4-triazole-3-carboxamide derivatives of the formula (V), as each defined in claim 1 or 2.

9. A pharmaceutical composition according to anyone of the claims 7 to 8, wherein the at least one compound with CB₁-receptor activity of formula (I), (II), (III), (IV) and/or (V), or a prodrug, tautomer or salt thereof, is present in an amount effectively suited for pediatric treatment and/or prophylaxis pertaining to psychiatric disorders such as psychosis, anxiety, depression, attention deficits, memory disorders, cognitive disorders, appetite disorders, obesity, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischemia, cerebral apoplexy, craniocerebral trauma, stroke, spinal cord injury, neuroinflammatory disorders, plaque sclerosis, viral encephalitis, demyelinisation related disorders, as well as for the pediatric treatment of pain disorders, including neuropathic pain disorders, and other diseases involving cannabinoid neurotransmission, including the pediatric treatment of septic shock, glaucoma, cancer, diabetes, emesis, nausea, asthma, respiratory diseases, gastrointestinal disorders, gastric ulcers, diarrhea and cardiovascular disorders, in a juvenile patient in need of such treating.

10. A pharmaceutical composition according to anyone of the claims 7 to 9, wherein the at least one compound with CB₁-receptor activity of formula (I), (II), (III), (IV) and/or (V), or a prodrug, tautomer or salt thereof, preferably the CB₁ receptor antagonistic compound or a prodrug, tautomer or salt thereof, is present in an amount effectively suited for the treatment and/or prophylaxis of obesity in juvenile patients and/or drug induced obesity in juvenile, as well as adolescent, patients.

11. A pharmaceutical composition containing as active components at least one compound with CB₁-receptor activity as defined in claim 2, or a prodrug, tautomer or salt thereof, preferably a CB₁ receptor antagonistic compound or a prodrug, tautomer or salt thereof, and at least one lipase inhibiting compound for the treatment and/or prophylaxis of obesity in adolescent or in juvenile patients and/or drug induced obesity in juvenile, as well as adolescent, patients.

12. A pharmaceutical composition according to claim 11, containing the at least one compound with CB₁-receptor activity or a prodrug, tautomer or salt thereof, preferably the CB₁ receptor antagonistic compound or a prodrug, tautomer or salt thereof, in combination with at least one lipase inhibiting compound selected from the group of lipase inhibiting polymers, orlistat, panclicins, ATL-962 and lipstatin.

13. A pharmaceutical composition according to any of the claims 10 to 12, wherein the at least one compound with CB₁-receptor activity, or a prodrug, tautomer or salt thereof, preferably the CB₁ antagonistic compound having a formula (I), (II), (III), (IV) or (V) as defined in claim 2, or the prodrug, tautomer or salt thereof, and the at least one lipase inhibiting compound each are present in an amount effectively suited for the treatment and/or prophylaxis of obesity in a juvenile patient in need of such treating.

14. A pharmaceutical composition according to any of the claims 10 to 12, wherein the at least one compound with CB₁-receptor activity, or a prodrug, tautomer or salt thereof, preferably the CB₁ antagonistic compound having a formula (I), (II), (III), (IV) or (V) as defined in claim 2, or the prodrug, tautomer or salt thereof, and the at least one lipase inhibiting compound each are present in an amount effectively suited for the treatment and/or prophylaxis of drug induced obesity in juvenile, as well as adolescent, patients in need of such treating.

15. A method of treatment and/or prophylaxis of CB₁ receptor related diseases in juvenile patients and/or for the treatment and/or prophylaxis of drug induced obesity in juvenile, as well as in adolescent, patients, characterized in that at least one compound with CB₁-receptor activity of formula (I), (II), (III), (IV) and/or (V), a prodrug thereof, a tautomer thereof or a salt thereof, as defined in claim 2 is administered to a patient in need of such treating.

16. A method of treatment and/or prophylaxis according to claim 15, wherein treatment and/or prophylaxis is directed to a pediatric treatment and/or prophylaxis pertaining to psychiatric disorders such as psychosis, anxiety, depression, attention deficits, memory disorders, cognitive disorders, appetite disorders, obesity, addiction, appetence, drug dependence and neurological disorders such as neurodegenerative disorders, dementia, dystonia, muscle spasticity, tremor, epilepsy, multiple sclerosis, traumatic brain injury, stroke, Parkinson's disease, Alzheimer's disease, epilepsy, Huntington's disease, Tourette's syndrome, cerebral ischemia, cerebral apoplexy, craniocerebral trauma, stroke, spinal cord injury, neuroinflammatory disorders, plaque sclerosis, viral encephalitis, demyelinisation related disorders, as well as for the pediatric treatment of pain disorders, including neuropathic pain disorders, and other diseases involving cannabinoid neurotransmission, including the pediatric treatment of septic shock, glaucoma, cancer, diabetes, emesis, nausea, asthma, respiratory diseases, gastrointestinal disorders, gastric ulcers, diarrhea and cardiovascular disorders.

17. A method of treatment and/or prophylaxis according to anyone of the claims 15 or 16, wherein the treatment and/or prophylaxis is directed to obesity in juvenile patients and/or drug induced obesity in juvenile, as well as adolescent, patients.

18. A method of treatment and/or prophylaxis of obesity in adolescent or in juvenile patients and/or of drug induced obesity in juvenile, as well as adolescent, patients, characterized in that at least one compound with CB₁-receptor activity as defined in claim 2, or a prodrug, tautomer or salt thereof, preferably a CB₁ receptor antagonistic compound or a prodrug, tautomer or salt thereof, is administered in combination with at least one lipase inhibiting compound to a patient in need of such treating.

19. A method of treatment and/or prophylaxis according to claim 18, characterized in that the at least one compound with CB₁-receptor activity or a prodrug, tautomer or salt thereof, preferably the CB₁ receptor antagonistic compound or a prodrug, tautomer or salt thereof, is administered in combination with at least one lipase inhibiting compound selected from the group of lipase inhibiting polymers, orlistat, panclicins, ATL-962 and lipstatin.

20. A method of treatment and/or prophylaxis according to any of the claims 15 to 19, characterized in that the treating is directed to obesity in juvenile patients.

21. A method of treatment and/or prophylaxis according to any of the claims 15 to 19, characterized in that the treating is directed to drug induced obesity in juvenile or adolescent patients.

22. A method of treatment and/or prophylaxis according to any of the claims 15 to 21, characterized in that the compound with CB₁-receptor activity or a prodrug, tautomer or salt thereof, preferably the CB₁ receptor antagonistic compound or a prodrug, tautomer or salt thereof, is administered in combination with the lipase inhibiting compound by simultaneous, separate or step-wise administration route.

23. Pharmaceutical product containing as a medicament at least one compound with CB₁-receptor activity having formula (I), (II), (III), (IV) or (V) as defined in claim 2, or a prodrug, tautomer or salt thereof, preferably a CB₁ receptor antagonistic compound or a prodrug, tautomer or salt thereof, as a combination preparation with at least one lipase inhibiting compound for simultaneous, separate or step-wise administration in the treatment and/or prophylaxis of obesity.

24. Pharmaceutical product containing as a medicament at least one compound with CB₁-receptor activity having formula (I), (II), (III), (IV) or (V) as defined in claim 2, or a prodrug, tautomer or salt thereof, preferably a CB₁ receptor antagonistic compound or a prodrug, tautomer or salt thereof, and a leaflet indicating that said compound with CB₁-receptor activity, preferably the CB₁ antagonistic compound, may be administered in combination with a lipase inhibiting compound for simultaneous, separate or step-wise administration in the treatment and/or prophylaxis of obesity.

25. A compound with CB₁-receptor activity having one of the formulas (I), (II), (III), (IV) or (V) as defined in claim 2, or a prodrug, tautomer or salt thereof, preferably of a CB₁ receptor antagonistic compound or a prodrug, tautomer or salt thereof, in combination with at least one lipase inhibiting compound.

26. A combination according to claim 25 of a compound with CB₁-receptor activity having one of the formulas (I), (II), (III), (IV) or (V) as defined in claim 2, wherein the compound with CB₁-receptor activity or a prodrug, tautomer or salt thereof, preferably the CB₁ receptor antagonistic compound or a prodrug, tautomer or salt thereof, is in combination with at least one lipase inhibiting compound selected from the group of lipase inhibiting polymers, orlistat, panclicins, ATL-962 and lipstatin.
